(19) United States
(12) Patent Application Publication
(10) Pub. No.: US 2011/0105381 A2
(43) Pub. Date: May 5, 2011

(54) Prodrugs of Peripheral Phenolic Opioid Antagonists

(75) Inventors: Thomas Jenkins, Half Moon Bay, CA (US); Aleksandr Kolesnikov, San Francisco, CA (US)

(73) Assignee: PharmacoFore, Inc., San Carlos, CA (US)

(21) Appl. No.: 12/524,544

(22) PCT Filed: Feb. 15, 2008
(86) PCT No.: PCTUS2008054127

§ 371 Date: Mar. 25, 2010

Related U.S. Application Data
(63) Continuation of application No. PCTUS2007069683, filed on May 24, 2007, now abandoned.
(60) Provisional application No. 60/901,795, filed on Feb. 16, 2007.
(60) Provisional application No. 60/990,203, filed on Nov. 26, 2007.
(60) Provisional application No. 60/901,795, filed on Feb. 16, 2007.

(51) Int. Cl. A61K 31/485; A61K 38/05; A61P 25/04; A61P 25/00; C07D 489/02; C07D 489/12

(52) U.S. Cl. 514/1.3; 514/282; 514/279; 546/45; 546/39

(57) ABSTRACT

Compounds of formula (I) 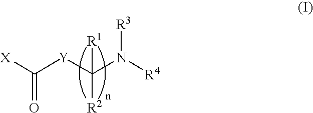
in which X, Y, R¹, R², n, R³ and R⁴ have the meanings given in the specification, are useful as pro-drugs of peripheral phenolic opioid antagonists.

The present invention relates to novel pro-drugs of peripheral phenolic opioid antagonists, to pharmaceutical compositions comprising the pro-drugs, to a process for making the pro-drugs and to the use of the pro-drugs, for example for countering the peripheral side effects of opioids in opioid therapy.

Opioids, for example hydrocodone or the phenolic opioids, morphine, hydromorphone or oxymorphone, are widely used in the treatment of pain. However, there are problems associated with their use.

One problem associated with the use of opioids is that they can cause unwanted effects that are partly or wholly peripherally mediated, such as constipation, cough suppression, dry mouth, heartburn, myocardial depression, nausea, pruritus, urinary retention, vomiting, bloating, dry-mouth or heartburn, by acting on the peripheral nervous system. These side effects can be countered by co-administering a peripheral opioid antagonist, such as the peripheral phenolic opioid antagonist, N-methylnaltrexone. Commonly the antagonists contain a bridgehead quaternary ammonium group, where the opioids have a bridgehead amino group. The selective action of these antagonists for peripheral opioid receptors arises from their poor ability to cross the blood brain barrier. Consequently, these peripheral opioid antagonists are also poorly absorbed through the gastrointestinal tract, and therefore need to be administered by injection. There is therefore a need for compounds that can be administered orally for use in providing patients with peripheral opioid antagonist treatment.

Delivery systems are often essential in safely administering active agents such as drugs. Often delivery systems can optimize bioavailability, improve dosage consistency and improve patient compliance (e.g., by reducing dosing frequency). Solutions to drug delivery and/or bioavailability issues in pharmaceutical development include converting known drugs to pro-drugs. Typically, in a pro-drug, a polar functional group (e.g., a carboxylic acid, an amino group, phenol group, a sulfhydryl group, etc.) of the active agent is masked by a promoiety, which is labile under physiological conditions. Accordingly, pro-drugs are usually transported through hydrophobic biological barriers such as membranes and may possess superior physicochemical properties in comparison to the parent drug. Pro-drugs are usually non-toxic and are ideally selectively cleaved at the locus of drug action. Preferably, cleavage of the promoiety occurs rapidly and quantitatively with the formation of non-toxic by-products (i.e., the hydrolyzed promoiety).

It has now been found that a pro-drug of the peripheral phenolic opioid antagonist, N-methylnaltrexone configured in a particular way can be administered orally. The compound has the chemical name (R)—N-methylnaltrexone 3-(N-methyl-N-(2-aminoethyl))carbamate. Accordingly, a way has now been found for providing patients with control of the peripheral side effects of an opioid by oral therapy. Certain derivatives of the pro-drug bearing a peptide residue on the terminal amino group have also been found to provide good systemic levels of the phenolic opioid antagonist when administered orally. Without wishing to be bound by theory, it is believed that these peptide derivatives may be undergoing cleavage by peptidases in the gut to afford the pro-drug, which then passes through the gut wall, releasing the phenolic opioid antagonist. Thus the technical effect of the pro-drug may be exploited either by orally administering the pro-drug itself, or a derivative thereof capable of delivering the pro-drug into the gut (a pro-drug of the pro-drug). It has also been found that this technical effect is not limited to the one peripheral phenolic opioid antagonist. It has also been observed for N-methylnaloxone.

According to one aspect, the present invention provides a method of antagonising peripheral action of an opioid in a patient undergoing opioid treatment, which comprises orally administering to said patient an effective amount of a compound of formula (I) 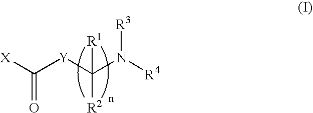
or a salt, hydrate or solvate thereof wherein:
X is a residue of a peripheral phenolic opioid antagonist, wherein the hydrogen atom of the phenolic hydroxyl group is replaced by a covalent bond to —C(O)—Y—(C(R¹)(R²))ₙ—N—(R³)(R⁴);
Y is —NR⁵—, —O— or —S—;
n is an integer from 1 to 10;
each R¹, R², R³ and R⁵ is independently hydrogen, alkyl, substituted alkyl, aryl or substituted aryl, or R¹ and R² together with the carbon to which they are attached form a cycloalkyl or substituted cycloalkyl group, or two R¹ or R² groups on adjacent carbon atoms, together with the carbon atoms to which they are attached, form a cycloalkyl or substituted cycloalkyl group; and
R⁴ is hydrogen, or a derivative thereof capable of delivering the compound of formula (I) into the gut.

Thus, in one embodiment, the present invention provides a compound of formula (I) in which R⁴ represents a hydrogen atom, or a salt, especially a pharmaceutically acceptable salt, thereof.

In another embodiment, the present invention provides a derivative of a compound of formula (I) capable of delivering the compound of formula (I) into the gut.

The derivative of the compound capable of delivering the compound of formula (I) into the gut may be any compound capable of conversion in the gut into a compound of formula I in which R⁴ represents hydrogen.

Compounds in which R⁴ is hydrogen and certain compounds corresponding with a compound of formula I in which R⁴ has been replaced with a peptidic residue have been found to afford improved systemic levels of phenolic opioid antagonist when administered orally to rats, for example an improved Cmax and/or longer detectable blood levels of phenolic opioid antagonist. Thus, compounds of formula (I) in which R⁴ is hydrogen or has been replaced with an acyl residue of the amino acid leucine or arginine have been found to afford good systemic levels of N-MTX when administered orally to rats. Compounds of formula (I) in which R⁴ has been replaced with an acyl residue of the amino acid aspartic acid, or a residue of the dipeptide glycylarginyl, N-acetylglycylarginyl or glycylphenylalanine, have been found to afford detectable levels of N-MTX for longer than when N-MTX itself is administered orally. Corresponding compounds containing acyl residues of other amino acids have so far been found to perform less favorably, but this may have been due to insufficient dosing of the compounds, or be a species dependent effect.

In general, it is believed that compounds capable of capable of delivering the compound of formula (I) into the gut may be selected from compounds of formula (I) in which X, Y, n, R¹, R² and R³ are as defined hereinabove and R⁴ represents 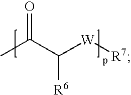
wherein:—
each R⁶ is independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, or optionally, R⁶ and R⁷ together with the atoms to which they are bonded form a cycloheteroalkyl or substituted cycloheteroalkyl ring;
R⁷ is hydrogen, alkyl, substituted alkyl, acyl, substituted acyl, alkoxycarbonyl, substituted alkoxycarbonyl, aryl, substituted aryl, arylalkyl or substituted arylalkyl;
p is an integer from 1 to 5;
each W is independently —NR⁸—, —O— or —S—; and
each R⁸ is independently hydrogen, alkyl, substituted alkyl, aryl or substituted aryl, or optionally, each R⁶ and R⁸ independently together with the atoms to which they are bonded form a cycloheteroalkyl or substituted cycloheteroalkyl ring.

The enzyme capable of cleaving the R⁴ group may be a peptidase—the enzymatically-cleavable moiety being linked to the nucleophilic nitrogen through an amide (e.g. a peptide: —NHCO—) bond. In some embodiments, the enzyme is a digestive enzyme such as, for example, pepsin, trypsin, chymotrypsin, colipase, elastase, aminopeptidase N, aminopeptidase A, dipeptidylaminopeptidase IV, tripeptidase or enteropeptidase.

It will be appreciated that when W is NH and R⁷ is H or acyl, then R⁴ is a residue of an amino acid or peptide, or an N-acyl derivative thereof. When W is NR⁸, R⁷ is H or acyl and R⁶ and R⁸ together with the atoms to which they are bonded form a pyrrolidine ring, then R⁴ is a residue of proline or an N-acyl derivative thereof.

Accordingly, in another embodiment, R⁴ is a residue of a D or L-amino acid (such as an L-amino acid) selected from alanine, arginine, asparagine, aspartic acid, cysteine, glycine, glutamine, glutamic acid, histidine, isoleucine, leucine, methionine, phenylalanine, proline, serine, threonine, tryptophan, tyrosine, lysine and valine; a residue of a dipeptide or tripeptide composed of two or three D or L amino acid residues (such as L-amino acid residues) selected independently from alanine, arginine, asparagine, aspartic acid, cysteine, glycine, glutamine, glutamic acid, histidine, isoleucine, leucine, methionine, phenylalanine, proline, serine, threonine, tryptophan, tyrosine, lysine and valine; or a residue of an N-acyl derivative thereof, such as an N-acetyl derivative.

In one embodiment, each of R¹, R², R³ and R⁵ is independently hydrogen, alkyl, substituted alkyl, aryl or substituted aryl.

In another embodiment, R⁶ is a side atom or group of a natural amino acid, such as H (from glycine), —CH₃ (from alanine), —CH₂CH(CH₃)₂ (from leucine), —CH₂(CH₂)₃NH₂ (from lysine), —CH₂CH₂CH₂NHC(NH)NH₂ (from arginine), 4-hydroxybenzyl (from tyrosine), CH₂COOH (from aspartic acid), —CH₂C(═O)NH₂ (from asparagine), or CH₂CH₂COOH (from glutamic acid). In another embodiment, R⁶ is benzyl (from phenylalanine).

In another embodiment, R⁷ is a hydrogen atom, or an unsubstituted of substituted acyl group, for example (1-6C)alkanoyl, such as acetyl or t-butanoyl; benzoyl unsubstituted or substituted by methylenedioxy or one or two substituents selected from (1-4C)alkyl, (1-4C)alkoxy or halogen, such as benzoyl or piperonyl; CONRₓR_{y} in which Rₓ and R_{y} are each independently hydrogen or (1-4C)alkyl, such as CONH₂), or a hemiacid or hemiester, such as CH₂CH₂COOH or CH₂CH₂COOEt. The unsubstituted of substituted acyl group is conveniently the residue of a pharmaceutically acceptable carboxylic acid.

Examples of particular values are:—

for Y: —NR⁵;

for R⁵: (1-4C)alkyl, such as —CH₃;

for R¹ and R²: hydrogen or (1-4C)alkyl, such as CH₃; more particularly hydrogen;

for n: 2 or 3;

for R³: hydrogen or (1-4C)alkyl, such as —CH₃;

for W: NH;

for R⁶: H (from glycine), —CH₃ (from alanine), —CH₂CH(CH₃)₂ (from leucine), —CH₂(CH₂)₃NH₂ (from lysine), —CH₂CH₂CH₂NHC(NH)NH₂ (from arginine), —CH₂C(═O)NH₂ (from asparagine), —CH₂COOH (from aspartic acid), —CH₂(p-hydroxyphenyl) (from tyrosine) or CH₂CH₂COOH (from glutamic acid), or CH₂(phenyl) (from phenylalanine), (such as H, —CH₂(CH₂)₃NH₂, —CH₂CH₂CH₂NHC(NH)NH₂, 4-hydroxybenzyl, CH₂COOH or CH₂CH₂COOH);

for R⁷: hydrogen, (1-6C)alkanoyl, such as acetyl or t-butanoyl, or optionally substituted benzoyl, for example benzoyl unsubstituted or substituted by methylenedioxy or one or two substituents selected from (1-4C)alkyl, (1-4C)alkoxy or halogen, such as benzoyl or piperonyl; in particular hydrogen or acetyl;

for a cycloheteroalkyl or substituted cycloheteroalkyl ring formed by R⁶ and R⁸ together with the atoms to which they are bonded: pyrrolidinyl;

for p: 1 or 2;

for R⁴: arginine, N-acetylarginine, N-t-butanoylarginine, N-benzoylarginine, N-piperonylarginine, N-glycinylarginine, N-acetylglycinylarginine, alanine, N-acetylalanine, asparagine, N-acetylasparagine, aspartic acid, N-acetylaspartic acid, lysine, N-acetyl]ysine, leucine, N-acetylleucine, glutamic acid, tyrosine, N-acetyltyrosine, proline or N-glycinylproline, or N-glycinylalanine or N-glycinylphenylalanine, (such as arginine, N-acetylarginine, N-t-butanoylarginine, N-benzoylarginine, N-piperonylarginine, N-glycinylarginine, lysine, glutamic acid, aspartic acid, tyrosine, proline and N-glycinylproline).

When R⁴ represents 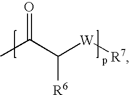
R³ preferably represents hydrogen.

The opioid may be, for example a phenolic opioid.

Phenolic opioids form a sub-group of the opioids, and include the widely prescribed drugs hydromorphone, oxymorphone, and morphine.

Specific examples of phenolic opioids include oxymorphone, hydromorphone, morphine and derivatives thereof. Particular mention is made of oxymorphone, hydromorphone and morphine. Other examples of phenolic opioids are buprenorphine, dihydroetorphine, diprenorphine, etorphine and levorphanol.

The peripheral phenolic opioid antagonist may be, for example, a quaternary ammonium salt, such as an N-methyl quaternary ammonium salt. It will be appreciated that the quaternary ammonium salt has an anion counter-ion. The counter-ion may be any pharmaceutically acceptable counter-ion, for example a chloride ion. Examples of peripheral phenolic opioid antagonists that are N-methyl quaternary ammonium salts are (R) N-methylnaltrexone, N-methylnaloxone, N-methyldiprenorphine, and N-methylnalmefene. In one embodiment, the peripheral opioid antagonist is (R)—N-methylnaltrexone (N-MTX). In another embodiment, the peripheral opioid antagonist is N-methylnaloxone (N-MNLX). It will be appreciated by those skilled in the art that (R)—N-methylnaltrexone antagonizes the actions of opioids such as hydromorphone, oxymorphone and morphine, but is limited in its ability to cross the blood brain barrier as compared to its tertiary amine analog. It therefore antagonizes only their peripheral actions, which are undesirable, not their actions on the central nervous system, such as pain relief, which are desirable. In one embodiment, the pro-drug of (R)—N-methylnaltrexone is a compound of formula (I) in which X represents the phenolic residue of (R)—N-methylnaltrexone, Y, R¹, R², n, R³ have any of the meanings given hereinabove, and R⁴ is hydrogen or has any of the meanings given hereinabove. Such a pro-drug may be administered orally. It will be appreciated that the parent drug, (R)—N-methylnaltrexone has poor oral bioavailability, and generally needs to be administered parenterally. Thus, the pro-drugs of (R)—N-methylnaltrexone in accordance with the present invention are useful whenever oral (R)—N-methylnaltrexone therapy is desired.

In another aspect the present invention provides a compound of formula I 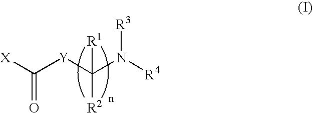
or a salt, hydrate or solvate thereof wherein:

X is (R)—N-methylnaltrexone, N-methylnaloxone, N-methyldiprenorphine or N-methylnalmefene, wherein the hydrogen atom of the phenolic hydroxyl group is replaced by a covalent bond to —C(O)—Y—(C(R¹)(R²))ₙ—N—(R³)(R⁴); and Y, R¹, R², n, R³ and R⁴ have any of the meanings given hereinabove. In a particular embodiment, X is (R)—N-methylnaltrexone, wherein the hydrogen atom of the phenolic hydroxyl group is replaced by a covalent bond to —C(O)—Y—(C(R¹)(R²))ₙ—N—(R³)(R⁴). In another embodiment, X is N-methylnaloxone, wherein the hydrogen atom of the phenolic hydroxyl group is replaced by a covalent bond to —C(O)—Y—(C(R¹)(R²))ₙ—N—(R³)(R⁴).

In another aspect, pharmaceutical compositions are provided which generally comprise one or more compounds of Formula (I), salts, hydrates or solvates thereof and a pharmaceutically acceptable vehicle such as a diluent, carrier, excipient or adjuvant. The choice of diluent, carrier, excipient and adjuvant will depend upon, among other factors, the desired mode of administration.

In still another aspect, methods for treating or preventing various diseases or disorders are provided. The methods generally involve administering to a patient in need of such treatment or prevention a therapeutically effective amount of a compound Formula (I) and/or a pharmaceutical composition thereof.

### BRIEF DESCRIPTION OF THE DRAWINGS

FIG. 1 shows the plasma concentration time course of the production of N-MTX following oral (PO) dosing of a compound of the present invention in rats.

FIG. 2 shows the plasma concentration time courses of the production of N-MTX following oral (PO) dosing of additional compounds of the present invention in rats.

FIG. 3 shows the plasma concentration time course of the production of N-MNLX following oral (PO) dosing of a compound of the present invention in rats.

### DETAILED DESCRIPTION

As used herein, the term “alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl.

In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Acyl” by itself or as part of another substituent refers to a radical —C(O)R³⁰, where R³⁰ is hydrogen, alkyl, cycloalkyl, cycloheteroalkyl, aryl, arylalkyl, heteroalkyl, heteroaryl, heteroarylalkyl as defined herein. Representative examples include, but are not limited to formyl, acetyl, t-butanoyl, cyclohexylcarbonyl, cyclohexylmethylcarbonyl, benzoyl, piperonyl, benzylcarbonyl and the like.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR³¹ where R³¹ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some embodiments, an aryl group comprises from 6 to 20 carbon atoms. In other embodiments, an aryl group comprises from 6 to 12 carbon atoms. Examples of an aryl group are phenyl and naphthyl.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenyleth-1-yl, naphthylmethyl, 2-naphthyleth-1-yl, naphthobenzyl, 2-naphthophenyleth-1-yl and the like. In some embodiments, an arylalkyl group is (C₇-C₃₀) arylalkyl, e.g., the alkyl moiety of the arylalkyl group is (C₁-C₁₀) and the aryl moiety is (C₆-C₂₀). In other embodiments, an arylalkyl group is (C₇-C₂₀) arylalkyl, e.g., the alkyl moiety of the arylalkyl group is (C₁-C₈) and the aryl moiety is (C₆-C₁₂).

Compounds may be identified either by their chemical structure and/or chemical name. The compounds described herein may contain one or more chiral centers and/or double bonds and therefore, may exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms. Certain compounds may exist in multiple crystalline or amorphous forms. In general, all physical forms are equivalent for the uses contemplated herein and are intended to be within the scope of the present disclosure.

“Cycloalkyl” by itself or as part of another substituent refers to a saturated cyclic alkyl radical. Typical cycloalkyl groups include, but are not limited to, groups derived from cyclopropane, cyclobutane, cyclopentane, cyclohexane and the like. In some embodiments, the cycloalkyl group is (C₃-C₁₀) cycloalkyl. In other embodiments, the cycloalkyl group is (C₃-C₇) cycloalkyl.

“Cycloheteroalkyl” by itself or as part of another substituent, refers to a saturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl” by themselves or as part of another substituent refer to alkyl groups, in which one or more of the carbon atoms (and any associated hydrogen atoms) are independently replaced with the same or different heteroatomic groups. Typical heteroatomic groups which can be included in these groups include, but are not limited to, —O—, —S—, —O—O—, —S—S—, —O—S—, —NR³⁷R³⁸—, ═N—N═, —N═N—, —N═N—NR³⁹R⁴⁰, —PR⁴¹—, —P(O)₂—, —POR⁴²—, —O—P(O)₂—, —SO—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group is from 5-20 membered heteroaryl. In other embodiments, the heteroaryl group is from 5-10 membered heteroaryl. In still other embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. In some embodiments, the heteroarylalkyl group is a 6-30 membered heteroarylalkyl, e.g., the alkyl moiety of the heteroarylalkyl is 1-10 membered and the heteroaryl moiety is a 5-20-membered heteroaryl. In other embodiments, the heteroarylalkyl group is 6-20 membered heteroarylalkyl, e.g., the alkyl moiety of the heteroarylalkyl is 1-8 membered and the heteroaryl moiety is a 5-12-membered heteroaryl.

“Opioid” refers to a chemical substance that exerts its pharmacological action by interaction at opioid receptors, providing patients with relief from pain. Examples of opioids include (3R,4S,beta-S)-13-fluoro ohmefentanyl, alfentanil, buprenorphine, carfentanil, codeine, diacetylmorphine, dihydrocodeine, dihydroetorphine, diprenorphine, etorphine, fentanyl, hydrocodone, hydromorphone, LAAM, levorphanol, lofentanil, meperidine, methadone, morphine, beta-hydroxy 3-methylfentanyl, oxycodone, oxymorphone, propoxyphene, remifentanil, sufentanil, tilidine and tramadol. “Phenolic opioid” refers to a subset of the opioids that contains a phenol group. Examples of phenolic opioids include buprenorphine, dihydroetorphine, diprenorphine, etorphine, hydromorphone, levorphanol, morphine, and oxymorphone. An “opioid antagonist” is a compound that antagonizes the pharmacological action of an opioid. The term includes phenolic opioid antagonists. Examples of phenolic opioid antagonists include naltrexone, naloxone, nalmefene, and (R)—N-methylnaltrexone. A “peripheral opioid antagonist” is a compound that is not capable of penetrating the blood/brain barrier or has a greatly reduced ability to cross the blood brain barrier compared to its tertiary amine analog, and hence is capable of antagonizing the (undesired) action of an opioid outside the central nervous system. An example of a peripheral phenolic opioid antagonist is (R)—N-methylnaltrexone. Other examples are N-methylnaloxone, N-methyldiprenorphine and N-methylnalmefene.

“Parent Aromatic Ring System” by itself or as part of another substituent, refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System” by itself or as part of another substituent, refers to a parent aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Pharmaceutical composition” refers to at least one compound and a pharmaceutically acceptable vehicle, with which the compound is administered to a patient.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with, or in which a compound is administered.

“Patient” includes humans, but also other mammals, such as livestock, zoo animals and companion animals.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Pro-drug” refers to a derivative of an active agent that requires a transformation within the body to release the active agent. Pro-drugs are frequently, although not necessarily, pharmacologically inactive until converted to the active agent.

“Promoiety” refers to a form of protecting group that when used to mask a functional group within an active agent converts the active agent into a pro-drug. Typically, the promoiety will be attached to the drug via bond(s) that are cleaved by enzymatic or non-enzymatic means in vivo.

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry,” (Wiley, 2^{nd} ed. 1991) and Harrison et al., “Compendium of Synthetic Organic Methods,” Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Substituted” refers to a group in which one or more hydrogen atoms are independently replaced with the same or different substituent(s). Typical substituents include, but are not limited to, alkylenedioxy (such as methylenedioxy), -M, —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In some embodiments, substituents include -M, —R⁶⁰, ═O, OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —NR⁶²C(O)NR⁶⁰R⁶¹. In other embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O⁻. In still other embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O⁻, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group.

“Treating” or “treatment” of any disease or disorder refers, in some embodiments, to ameliorating the disease or disorder (i.e., arresting or reducing the development of the disease or at least one of the clinical symptoms thereof). In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter), or both. In still other embodiments, “treating” or “treatment” refers to delaying the onset of the disease or disorder.

“Therapeutically effective amount” means the amount of a compound that, when administered to a patient for treating a disease, is sufficient to effect such treatment for the disease. The “therapeutically effective amount” will vary depending on the compound, the disease and its severity and the age, weight, etc., of the patient to be treated.

The compounds of formula (I) described herein in which R⁴ represents 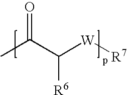
may be obtained via the routes generically illustrated in Schemes 1-4.

The promoieties described herein, may be prepared and attached to drugs containing phenols by procedures known to those of skill in the art (See e.g., Green et al., “Protective Groups in Organic Chemistry,” (Wiley, 2^{nd} ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-17, (Wiley Interscience); Trost et al., “Comprehensive Organic Synthesis,” (Pergamon Press, 1991); “Theilheimer's Synthetic Methods of Organic Chemistry,” Volumes 1-45, (Karger, 1991); March, “Advanced Organic Chemistry,” (Wiley Interscience), 1991; Larock “Comprehensive Organic Transformations,” (VCH Publishers, 1989); Paquette, “Encyclopedia of Reagents for Organic Synthesis,” (John Wiley & Sons, 1995), Bodanzsky, “Principles of Peptide Synthesis,” (Springer Verlag, 1984); Bodanzsky, “Practice of Peptide Synthesis,” (Springer Verlag, 1984). Further, starting materials may be obtained from commercial sources or via well established synthetic procedures, supra.

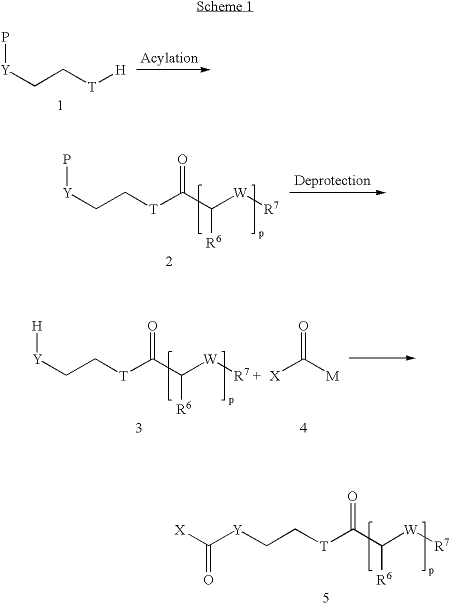

Referring now to Scheme 1 and formula I, supra, where for illustrative purposes T is NR³, Y is NR⁵, —O— or —S—, W is NR⁸, —O— or —S—, n is 2, R¹ and R² are hydrogen, p, R³, R⁵, R⁶, R⁷ and R⁸ are as previously defined, X is a peripheral phenolic opioid antagonist, P is a protecting group, and M is a leaving group, compound 1 may be acylated with an appropriate carboxylic acid or carboxylic acid equivalent to provide compound 2 which then may be deprotected to yield compound 3. Compound 3 is then reacted with an activated carbonic acid equivalent 4 to provide desired compound 5.

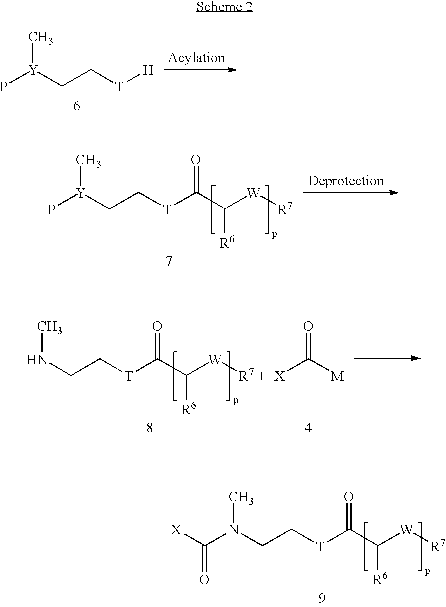

Referring now to Scheme 2 and formula I, supra, where for illustrative purposes T is NR³, Y is NCH₃, W is NR⁸, —O— or —S—, n is 2, R¹ and R² are hydrogen, p, R³, R⁶, R⁷ and R⁸ are as previously defined, X is a peripheral phenolic opioid antagonist, P is a protecting group, and M is a leaving group, compound 6 is acylated with an appropriate carboxylic acid or carboxylic acid equivalent to provide compound 7. Compound 7 is then deprotected and reacted with activated carbonic acid equivalent 4 to provide desired compound 9.

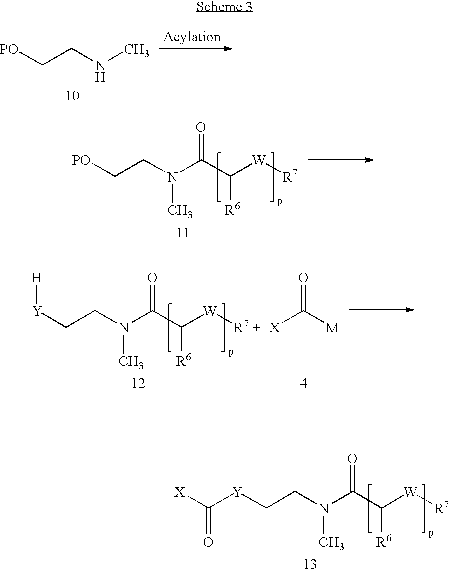

Referring now to Scheme 3 and formula I, supra, where for illustrative purposes T is NCH₃, Y is NR⁵, —O— or —S—, W is NR⁸, —O— or —S—, n is 2, R¹ and R² are hydrogen, p, R⁵, R⁶, R⁷ and R⁸ are as previously defined, X is a peripheral phenolic opioid antagonist, P is a protecting group, and M is a leaving group, compound 10 is acylated with an appropriate carboxylic acid or carboxylic acid equivalent to provide compound 11 which after deprotection and functional group intraconversion, if necessary, is converted to compound 12. Reaction of compound 12 with activated carbonic acid equivalent 4 provides desired compound 13.

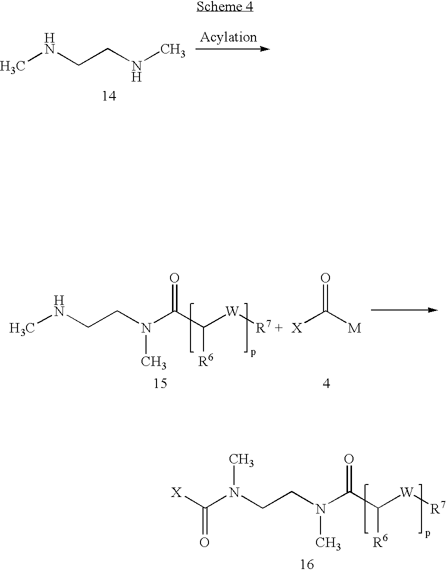

Referring now to Scheme 4 and formula I, supra, where for illustrative purposes T and Y are NCH₃, W is NR⁸, —O— or —S—, n is 2, R¹ and R² are hydrogen, p, R⁶, R⁷ and R⁸ are as previously defined, X is a peripheral phenolic opioid antagonist, P is a protecting group, and M is a leaving group, compound 14 is acylated with an appropriate carboxylic acid or carboxylic acid equivalent to provide compound 15. Reaction of compound 15 with activated carbonic acid equivalent 4 provides desired compound 16.

A compound of formula (I) so prepared in which R⁷ represents a hydrogen atom may then be further acylated to afford a corresponding compound of formula (I) in which the value of p has been increased, or in which R⁷ represents an acyl group.

The corresponding compounds of formula (I) in which R⁴ represents a hydrogen atom may be prepared in an analagous manner, starting from a corresponding starting material bearing an amino protecting group in place of 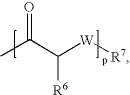
and ending with removal of the protecting group.

According to another aspect, therefore, the present invention provides a process for the preparation of a compound of formula (I) or a pharmaceutically acceptable salt thereof, which comprises reacting a compound of formula (III) 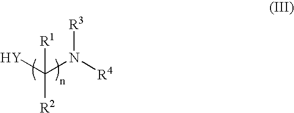
or a protected derivative thereof, with a compound of formula (IV) 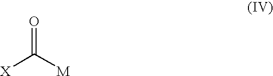
in which M represents a leaving atom or group, such as an activated aryloxycarbonyl group, for example p-nitrophenoxycarbonyl;

followed by removing any protecting groups and, if desired, acylating a compound of formula (I) in which R⁷ (in the group R⁴ as defined hereinabove) represents a hydrogen atom and/or forming a pharmaceutically acceptable salt.

Compounds of formula (I) in which X represents a residue of (R)—N-methylnaltrexone can also be prepared by methylating a corresponding compound of formula (I) in which X is a residue of naltrexone, or a protected derivative thereof. It will be appreciated that the desired (R) isomer may be separated from the (S) isomer by resolution techniques known in the art, for example using chiral phase chromatography.

(R)—N-methylnaltrexone is a known compound, which may be prepared by methylation of naltrexone, for example as described in WO2006127899. Other peripheral phenolic opioid antagonists that are quaternary ammonium salts may be prepared by alkylation of the bridgehead amine group in an analogous manner. N-Methyldiprenorphine and N-methylnalmefene, which may be prepared by methylation of diprenorphine and nalmefene respectively, are believed to be novel, and are provided as a further aspect of the invention.

Selection of appropriate protecting groups, reagents and reaction conditions for any of the steps in the above Schemes is well within the ambit of those of skilled in the art. Other methods for synthesis of the pro-drugs described herein will be readily apparent to the skilled artisan and may be used to synthesize the compounds described herein. Accordingly, the methods presented in the Schemes herein are illustrative rather than comprehensive.

The invention further provides all the novel intermediates described herein.

In general, the pro-drugs disclosed herein may be used to treat and/or prevent the same disease(s) and/or conditions as the parent drug which are well known in the art (see, e.g., Physicians Desk Reference, 2000 54^{th} Edition and the Merck Index, 13^{th} Edition). Phenolic opioids are useful in the treatment of pain.

For example, a phenolic opioid such as hydromorphone can be used, inter alia, to treat or prevent pain including, but not limited to include, acute pain, chronic pain, neuropathic pain, acute traumatic pain, arthritic pain, osteoarthritic pain, rheumatoid arthritic pain, muscular skeletal pain, post-dental surgical pain, dental pain, myofascial pain, cancer pain, visceral pain, diabetic pain, muscular pain, post-herpetic neuralgic pain, chronic pelvic pain, endometriosis pain, pelvic inflammatory pain and child birth related pain. Acute pain includes, but is not limited to, acute traumatic pain or post-surgical pain. Chronic pain includes, but is not limited to, neuropathic pain, arthritic pain, osteoarthritic pain, rheumatoid arthritic pain, muscular skeletal pain, dental pain, myofascial pain, cancer pain, diabetic pain, visceral pain, muscular pain, post-herpetic neuralgic pain, chronic pelvic pain, endometriosis pain, pelvic inflammatory pain and back pain.

A pro-drug of a peripheral phenolic opioid antagonist in accordance with the present invention can be used to antagonize the peripheral action of an opioid in a patient undergoing opioid treatment. Such a peripheral phenolic opioid antagonist pro-drug, when administered orally, has a superior bioavailability compared to its parent counterpart. For example, oral administration of such a periopheral phenolic opioid antagonist pro-drug can lead to enhanced concentrations (e.g., maximum concentrations) and/or enhanced persistence of exposure over time of the respective peripheral phenolic opioid antagonist in a patient compared to oral administration of the antagonist alone. A peripheral phenolic opioid pro-drug in accordance with the present invention can be administered to a patient undergoing therapy with any opioid agonist or partial agonist that causes peripheral side effects. In one embodiment, such a peripheral phenolic opioid pro-drug can be administered to a patient treated with post administration-activated, controlled release of a phenolic opioid.

The pharmaceutical compositions disclosed herein comprise a pro-drug disclosed herein with a suitable amount of a pharmaceutically acceptable vehicle, so as to provide a form for proper administration to a subject.

Suitable pharmaceutical vehicles include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions may be manufactured by means of conventional mixing, dissolving, granulating, dragee-making, levigating, emulsifying, encapsulating, entrapping or lyophilizing processes. Pharmaceutical compositions may be formulated in a conventional manner using one or more physiologically acceptable carriers, diluents, excipients or auxiliaries, which facilitate processing of compositions and compounds disclosed herein into preparations which can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions or any other form suitable for use known to the skilled artisan. In some embodiments, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 19th Edition, 1995).

Pharmaceutical compositions for oral delivery may be in the form of tablets, lozenges, aqueous or oily suspensions, granules, powders, emulsions, capsules, syrups, slurries, suspensions or elixirs, for example. Orally administered compositions may contain one or more optional agents, for example, sweetening agents such as fructose, aspartame or saccharin, flavoring agents such as peppermint, oil of wintergreen, or cherry coloring agents and preserving agents, to provide a pharmaceutically palatable preparation. Moreover, when in tablet or pill form, the compositions may be coated to delay disintegration and absorption in the gastrointestinal tract, thereby providing a sustained action over an extended period of time. Oral compositions can include standard vehicles such as mannitol, lactose, starch, magnesium stearate, sodium saccharine, cellulose, magnesium carbonate, sucrose, sorbitol, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP), granulating agents, binding agents and disintegrating agents such as the cross-linked polyvinylpyrrolidone, agar, or alginic acid or a salt thereof such as sodium alginate etc.

The amount of compounds disclosed herein and/or pharmaceutical compositions thereof that will be effective in the treatment or prevention of diseases in a patient will depend on the specific nature of the condition and can be determined by standard clinical techniques known in the art. The amount of compounds disclosed herein and/or pharmaceutical compositions thereof administered will, of course, be dependent on, among other factors, the subject being treated, the weight of the subject, the severity of the affliction, the manner of administration and the judgment of the prescribing physician.

In certain embodiments, compounds disclosed herein and/or pharmaceutical compositions thereof can be used in combination therapy with at least one other therapeutic agent. The compounds disclosed herein and/or pharmaceutical compositions thereof and the therapeutic agent can act additively or, more preferably, synergistically. In some embodiments, compounds disclosed herein and/or pharmaceutical compositions thereof are administered concurrently with the administration of another therapeutic agent. For example, compounds disclosed herein and/or pharmaceutical compositions thereof may be administered together with another therapeutic agent (e.g. including, but not limited to, laxatives, non-opioid analgesics and the like). In other embodiments, compounds disclosed herein and/or pharmaceutical compositions thereof are administered prior or subsequent to administration of other therapeutic agents.

It will be apparent to those skilled in the art that many modifications, both to materials and methods, may be practiced without departing from the scope of this disclosure. Accordingly, the present embodiments are to be considered as illustrative and not restrictive, and the invention is not to be limited to the details given herein, but may be modified within the scope and equivalents of the allowed claims.

All publications and patents cited herein are incorporated by reference in their entirety.

The following examples illustrate the invention.

In the examples, the following abbreviations are used:—

HOBt: 1-Hydroxybenzotriazole; PyBOP: Benzotriazole-1-yl-oxy-tris-pyrrolidino-phosphonium hexafluorophosphate; DIEA: diisopropylethylamine; and BocGlyOSu: N—(N-alpha-glycinyloxy)succinimide.

Amino acids in structures depicted in the examples are intended to be natural L amino acids. Quaternary salt structures are intended to be depicted in the R configuration.

### Preparation 1

Naltrexone free base was prepared according to a protocol similar to that described in U.S. Pat. No. 4,176,186.

(R)—N-methylnaltrexone was synthesized according to a protocol similar to that described in WO2006127899.

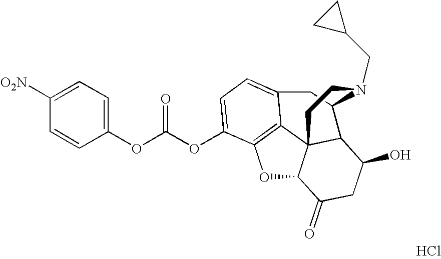

Naltrexone (0.34 g (1.0 mmol) was dissolved in dichloromethane (10 ml). p-Nitrophenylchlorocarbonate (0.212 g (1.1 mmol) in dichloromethane (5 ml) was then added dropwise over a period of 5 minutes. The reaction mixture was then sonicated for 2 hours to afford a stock solution of the depicted product that was used in the next step.

### Preparation 2

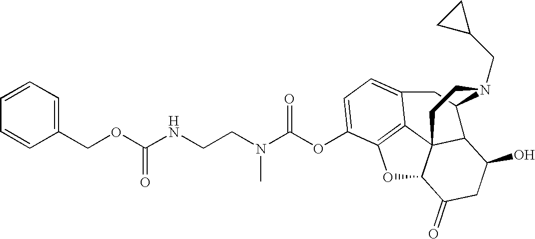

The product of Preparation 1 (stock solution, 15 ml, 1.0 mmol) was added to the solution of 0.265 g (1.05 mmol) of benzyl 2-(methylamino)ethylcarbamate hydrochloride in 10 ml of dimethylformamide. The pH was then adjusted by adding triethylamine (0.28 ml, 2.0 mmol). The reaction mixture was then stirred for 2 hours. The solvent was then evaporated under a vacuum, and the residue was dissolved in ethyl acetate (20 ml) and washed four times with 10 ml of 1M aqueous sodium carbonate. The organic layer was then washed three times with water (10 ml) and once with brine (10 ml), then dried over magnesium sulfate. The solvent was then removed by evaporation to afford the depicted product 0.425 g (74%). Mass spec: Calculated 575.26 Observed 576.4.

### Preparation 3

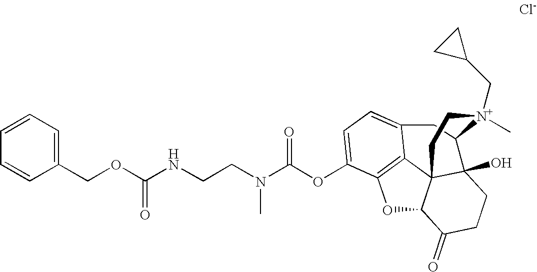

The product of Preparation 2 (0.425 g, 0.74 mmol) was dissolved in 5 ml of dry acetone. Methyl iodide (1.42 g, 10 mmol) was added and the mixture was heated in a capped tube at 85° C. for 3 days. The solvent was then removed by evaporation. The residue was then dissolved in 10 ml of methanol and loaded onto a column with 4 g of anion-exchange resin, chloride form (DOWEX 1×2-200). The chloride salt was eluted from the column using 50 ml of methanol. The solution was then evaporated to 10 ml volume and mixed with 2 g of silica gel. The remaining solvent was then evaporated and the residual dry powder was loaded onto a silica gel column. Remaining starting compound was then eluted with dichloromethane/1M solution of ammonia in methanol (95:5). The product was then eluted with dichloromethane/1M solution of ammonia in methanol (70:30) to afford the depicted compound 0.125 g (27%).

### Example 1

### (R)—N-Methylnaltrexone 3-(N-methyl-N-(2-aminoethyl))carbamate

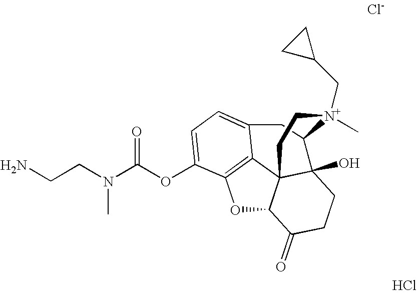

The product of Preparation 3 (0.125 g, 0.2 mmol) was dissolved in trifluoroacetic acid (3 ml). A 1 M solution of boron tribromide in dichloromethane (0.4 ml, 0.4 mmol) was added at 0-5° C. The mixture was then stirred for 2 hours. The solvent was removed in vacuum. 10 ml of 3 N aqueous hydrogen chloride were mixed with the residue and the mixture was stirred for 16 hours. After evaporation of water under a vacuum, the crude product was purified by reverse phase preparative HPLC (acetonitrile gradient) to afford the depicted compound (0.032 g, 30%). Mass spec: Calculated 456.25. Observed 456.4.

### Preparation 4

### 3-O-Isobutyryl-Naltrexone hydrochloride

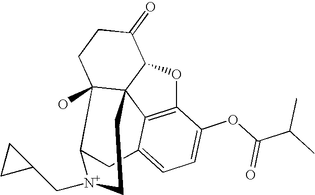

A mixture of Naltrexone hydrochloride (1) (3.78 g, 10 mmol) and isobutyric anhydride (3.2 g, 20 mmol) was heated at 90° C. in 100 ml dry dioxane overnight. The solvent and excess of reagents were evaporated under reduced pressure. The evaporation procedure was repeated after addition of xylenes (50 ml). The residue was used in the next step without purification. Yield: ˜4.5 g, (˜100%)

### Preparation 5

### 3-O-Isobutyryl-N-Methylnaltrexone Iodide/Chloride

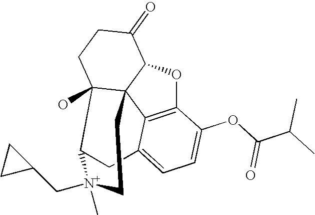

O-Isobutyryl-Naltrexone hydrochloride (2) from Preparation 4 (˜4.5 g, ˜10 mmol) and methyl iodide (10 g, ˜70 mmol) were placed in to 20 ml microwave tube. The reaction mixture was heated at 100° C. for 12 hour. LC MS analysis showed ˜90% conversion. The excess of reagents was then removed under reduced pressure. The residue was used in the next step without purification.

### Preparation 6

### N-Methylnaltrexone (N-MTX) Trifluoroacetate

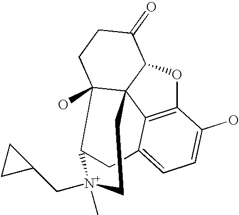

3-O-Isobutyryl-N-Methylnaltrexone Iodide/Chloride from Preparation 5 (˜10 mmol) was dissolved in 100 ml 50% MeOH followed by addition of 10 ml 48% HBr (aq.). The reaction mixture was kept at 60-70° C. overnight. LC-MS showed total hydrolysis. The volatiles were then removed under reduced pressure. The residual brown oil was purified by prep HPLC on RP-18 silica gel column (1.5 in, ×300). Fractions with N-MTX (>95%) were collected and evaporated. Yield: ˜2.5 g, (˜54%).

### Preparation 7

### 3-(4-Nitrophenyl)-N—(R)-Methylnaltrexone carbonate

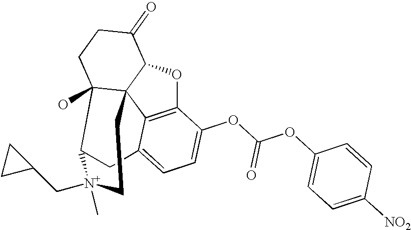

N-Methylnaltrexone (N-MTX) Trifluoroacetate (0.43 g, 0.9 mmol) and DIEA (0.2 ml, 1.2 mmol) were dissolved in mixture of 1 ml DMF and 20 ml CHCl₃ using an ultrasound bath. The solution was cooled down to 0° C. followed by addition of nitrophenylchloroformate (0.2 g, 1 mmol). The reaction mixture was sonicated 30 min at r.t. Conversion to p-nitrophenylcarbonate was monitored by LC-MS. The solution of 3-(4-Nitrophenyl)-N-Methylnaltrexone carbonate was used without change in the next step

### Preparation 8

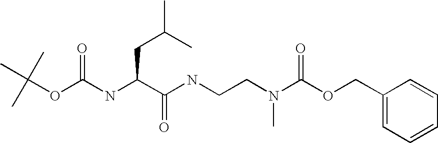

Boc-L-Leu-OH hydrate (3 mmol, 0.75 g), benzyl 2-(methylamino)ethylcarbamate hydrochloride (3.1 mmol 0.75 g) and BOP-reagent (3.1 mmol, 1.33 g) were dissolved in 25 ml DMF followed by addition of DIEA (1.2 ml, ˜7 mmol). The reaction mixture was stirred for 2 h at ambient temperature, then diluted with 100 ml ethyl acetate and transferred to a separatory funnel. The ethyl acetate layer was washed twice with water (2×150 ml), brine (100 ml), and dried over MgSO₄. The drying agent was then filtered off and the solvent was removed under reduced pressure. Yield: ˜1.2 g, (95%).

### Preparation 9

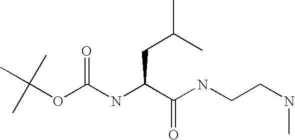

The product of Preparation 8 (1.2 g, 2.9 mmol) was dissolved in ˜30 ml MeOH, purged with nitrogen followed by addition of 5% Pd/C (˜150 mg). Hydrogenolysis was performed at 60 psi (about 414 kPa) during 2 h on a Parr's apparatus. The catalyst was then filtered off and the solvent was removed under reduced pressure to yield a clear oil. (0.84 g, ˜100%).

### Preparation 10

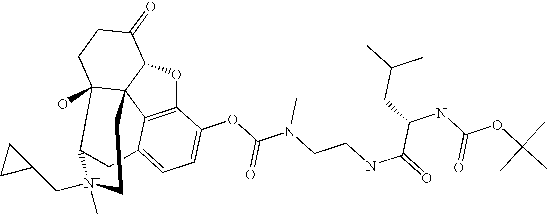

A solution of 3-(4-Nitrophenyl)-N—(R)-Methylnaltrexone carbonate from Preparation 7 in chloroform (10 ml, ˜0.45 mmol) was mixed with the product of Preparation 9 (7) (0.2 g, 0.6 mmol) and diluted with 5 ml DMF. Chloroform was removed under reduced pressure (bath temperature below 30° C.). The residual solution was kept at ambient temperature for 2 h. The reaction was monitored by LC MS. The product was purified by reverse phase HPLC on RP-18 silica gel column (1.5 in.×300, water-MeCN, 0.1% TFA). Fractions with the product were collected and evaporated (bath temperature below 40° C.), yielding a colorless oil. (˜0.3 g).

### Example 2

### (R)—N-Methylnaltrexone 3-(N-methyl-N-(2-leucinylamino))ethylcarbamate Hydrochloride

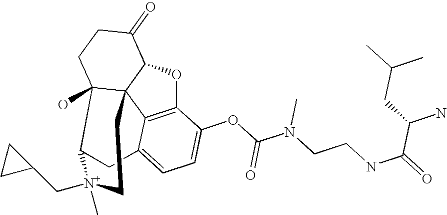

The product of Preparation 10 was treated 5 min with 5 ml TFA at ambient temperature. The acid was then removed by evaporation. The residual oil was dissolved in ˜4 ml AcOH, diluted with ˜15 ml 2M HCl in ether and 30 ml ether. Solid material was precipitated by centrifugation, treated with ether again and dried under high vacuum overnight. Yield: 0.17 g, (54%). Purity: 95%. Mass spec: Calculated 569.73. Observed 569.6.

### Preparation 11

### {2-[Fmoc-Arg(Pbf)]-aminoethyl}-methyl-carbamic acid benzyl ester

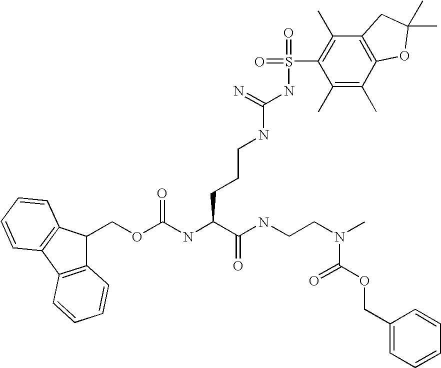

A solution of Fmoc-Arg(Pbf)-OH (5.01 g, 7.7 mmol), (2-amino-ethyl)-methyl-carbamic acid benzyl ester hydrochloride (2.09 g, 8.5 mmol), benzotriazol-1-yloxy)tris(dimethylamino)-phosphonium hexafluorophosphate (BOP) (4.1 g, 9.2 mmol), and DIEA (4.5 ml, 26.2 mmol) in dimethylformamide (DMF) (10 ml) was stirred at ambient temperature for 45 min followed by the dilution with ethyl acetate (EtOAc) (100 ml). The solution was washed with water (3 times using 30 ml each time (30 ml×3)) and brine (30 ml). The organic layer was dried over magnesium sulfate (MgSO₄) and evaporated to provide the title compound as yellowish oil. Mass spec: Calculated 839.03. Observed 839.3. The product was used as is for the next synthesis transformation.

### Preparation 12

### {2-[H-Arg(Pbf)]-aminoethyl}-methyl-carbamic acid benzyl ester

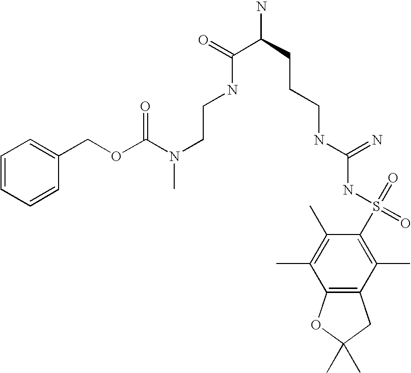

A mixture of the product of Preparation 11 (7.7 mmol) and piperidine (3.81 ml, 38.5 mmol) in ethyl acetate (50 ml) was maintained at ambient temperature for 40 min. The solvents were evaporated, and the residue was suspended in isopropanol (i-PrOH) (20 ml). The mixture was evaporated (procedure was repeated twice). The residue was dissolved in ethyl acetate (5 ml), and hexane was added (300 ml). The formed precipitate was filtrated and washed with hexane (procedure was repeated three times). The precipitate was dried in vacuum over night to provide the title compound (4.71 g, 99%) as yellowish solid. Mass spec: Calculated 617.8. Observed 617.3.

### Preparation 13

### {2-[Boc-Arg(Pbf)]-aminoethyl}-methyl-carbamic acid benzyl ester

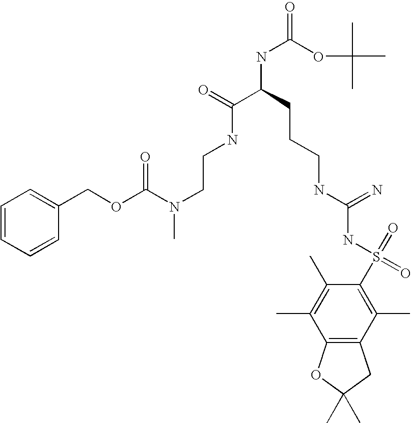

A solution of the product of Preparation 12 (1.19 g, 1.93 mmol), di-tert-butyl dicarbonate (Boc₂O) (5.04 g, 2.31 mmol) and DIEA (402 μl, 2.31 mmol) in dichloromethane (DCM) (7 ml) was maintained at ambient temperature for 2 h. followed by dilution with ethyl acetate (100 ml). The solution was extracted with water (30 ml×3) and brine (30 ml). The organic layer was dried over MgSO₄ and evaporated. The residue was dissolved in DCM (2 ml) and subjected to flash chromatography on CombiFlash® Companion unit equipped with RediSep (flash column (normal phase, 35-60 micron average particle size silicagel, 40 g, Teledyne Isco); flow rate=30 ml/min; injection volume 2 ml; mobile phase A: hexane; mobile phase B: EtOAc; gradient 0-100% B in 30 min. Fractions containing the desired product were combined and concentrated in vacuum to provide the title compound (1.13 g, 81%) as colorless oil. Mass spec: Calculated 717.9. Observed 717.3.

### Preparation 14

### Synthesis of N-[Boc-Arg(Pbf)]-N′-methyl-ethane-1,2-diamine

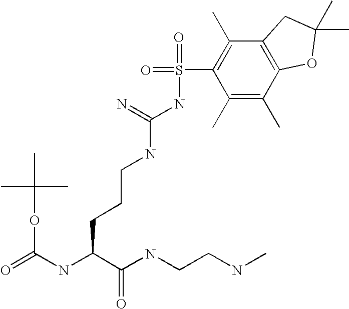

The product of Preparation 13 (1.13 g, 1.6 mmol) was dissolved in methanol (50 ml) followed by the addition of palladium on carbon (Pd/C) (5% wt, 1 g) suspension in water (2 ml). The reaction mixture was subjected to hydrogenation (Parr apparatus, 60 psi) at ambient temperature for 30 min. The catalyst was filtered and washed with methanol. The filtrate was evaporated in vacuum to provide the title compound (913 mg, 98%) as colorless oil. Mass spec: Calculated 583.8. Observed 583.2.

### Preparation 15

### Synthesis of Protected N-MTX Derivative

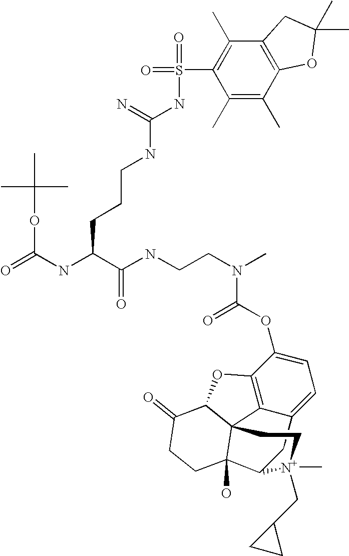

A suspension containing the trifluoroacetic acid (TFA) salt of N-MTX (329 mg, 0.7 mmol) and DIEA (122 μl, 0.7 mmol) in chloroform (4 ml) was sonicated in an ultrasonic bath at room temperature for 30 min followed by the addition of 4-nitrophenyl chloroformate (141 mg, 0.7 mmol). The reaction was sonicated in an ultrasonic bath at room temperature for 30 additional minutes followed by the addition of a solution of the product of Preparation 14 (466 mg, 0.8 mmol) and 1-hydroxybenzo-triazole (HOBt) (164 mg, 1.2 mmol) in DMF (3 ml). The reaction mixture was stirred overnight at ambient temperature. Volatile solvents were evaporated in vacuum. The residual solution was subjected to HPLC purification. [Nanosyn-Pack Microsorb (100-10) C-18 column (50×300 mm); flow rate=100 ml/min; injection volume 3 ml; mobile phase A: 100% water, 0.1% TFA; mobile phase B: 100% acetonitrile (ACN), 0.1% TFA; gradient elution from 0% B to 70% B in 70 min., detection 254 nm]. Fractions containing the desired compound were combined and concentrated in vacuum to provide the title compound as an off-white glass-like solid. Mass spec: Calculated 964.2. Observed 964.6.

### Example 3

### (R)—N-Methylnaltrexone 3-(N-methyl-N-(2-arginylamino))ethylcarbamate

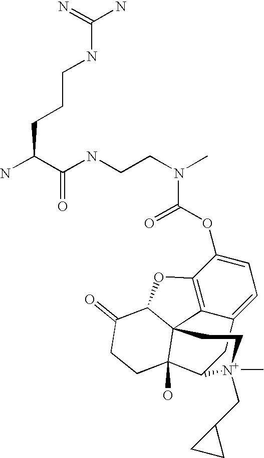

The product of Preparation 15 was dissolved in isopropanol (15 ml) and evaporated in vacuum. The residue was dissolved in a mixture of 5% m-cresol/TFA (10 ml). The reaction mixture was maintained at ambient temperature for 1 h followed by dilution with ethyl ester (100 ml). The formed precipitate was centrifuged, and supernatant was discharged (procedure was repeated twice). The precipitate was dissolved in water (5 ml) and subjected to HPLC purification. [Nanosyn-Pack Microsorb (100-10) C-18 column (50×300 mm); flow rate=100 ml/min; injection volume 3 ml; mobile phase A: 100% water, 0.1% TFA; mobile phase B: 100% ACN, 0.1% TFA; gradient elution from 0% B to 70% B in 70 min., detection 254 nm]. Fractions containing the desired compound were combined and concentrated in vacuum. The residue was dissolved in isopropanol (15 ml) and evaporated in vacuum to provide a TFA salt of the title compound as colorless glass-like solid. The title compound was dissolved in dioxane (2 ml) and 4 M HCl/dioxane (10 ml) was added. The reaction mixture was maintained at ambient temperature for 30 min. Solvents were evaporated. The residue was dried in vacuum to provide a hydrochloric salt of the title compound as a white solid. Yield: 110 mg (24%). Purity: 100%. Mass spec: Calculated 612.8. Observed 612.4.

### Preparation 16

### {2-[Ac-Arg(Pbf)]-aminoethyl}-methyl-carbamic acid benzyl ester

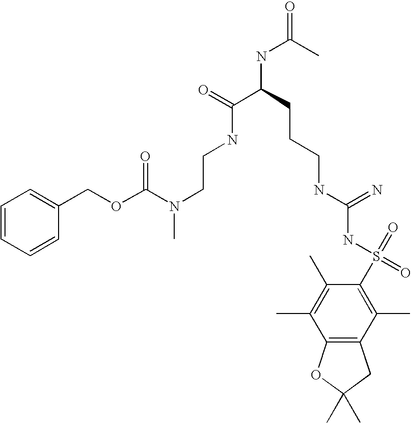

A solution of the product of Preparation 12 (1.19 g, 1.93 mmol), acetic anhydride (Ac₂O) (363 μl, 3.86 mmol) and DIEA (672 μl, 3.86 mmol) in DCM (7 ml) was maintained at ambient temperature for 2 h followed by the addition of 2 Methylamine/tetrahydrofuran (EtNH₂/THF) (3 ml, 5.79 mmol). The reaction mixture was stirred at ambient temperature for 30 additional min followed by dilution with ethyl acetate (100 ml). The solution was washed with water (30 ml×3) and brine (30 ml). The organic layer was dried over MgSO₄ and evaporated to provide the title compound (1.12 g, 88%) as yellowish oil. Mass spec: Calculated 659.8. Observed 659.4.

### Preparation 17

### N—[Ac-Arg(Pbf)]-N′-methyl-ethane-1,2-diamine

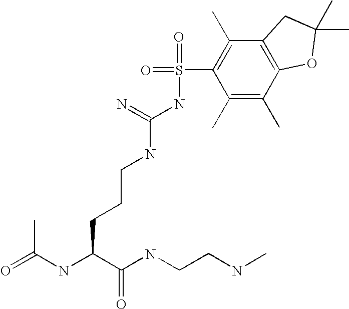

The product of Preparation 16 (1.12 g, 1.7 mmol) was dissolved in methanol (50 ml) followed by the addition of Pd/C (5% wt, 1 g) suspension in water (2 ml). The reaction mixture was subjected to hydrogenation (Parr apparatus, 60 psi) at ambient temperature for 30 min. The catalyst was filtered and washed with methanol. The filtrate was evaporated in vacuum to provide the title compound (882 mg, 99%) as colorless oil. Mass spec: Calculated 525.7. Observed 525.3.

### Preparation 18

### Synthesis of Protected N-MTX Derivative

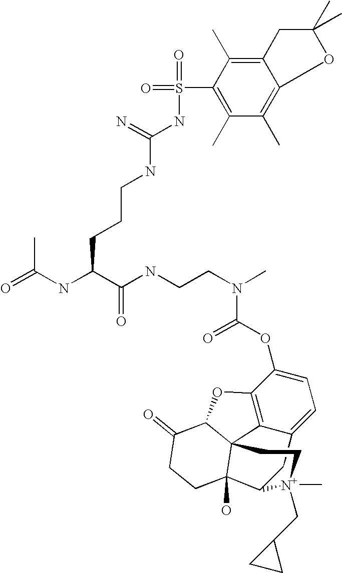

A suspension containing the TFA salt of N-MTX (329 mg, 0.7 mmol) and DIEA (122 μl, 0.7 mmol) in chloroform (4 ml) was sonicated in an ultrasonic bath at room temperature for 30 min followed by the addition of 4-nitrophenyl chloroformate (141 mg, 0.7 mmol). The reaction was sonicated in an ultrasonic bath at room temperature for 30 additional minutes followed by the addition of a solution of the product of Preparation 17 (419 mg, 0.8 mmol) and 1-hydroxybenzo-triazole (164 mg, 1.2 mmol) in DMF (3 ml). The reaction mixture was stirred overnight at ambient temperature. Volatile solvents were evaporated in vacuum. The residual solution was subjected to HPLC purification. [Nanosyn-Pack Microsorb (100-10) C-18 column (50×300 mm); flow rate=100 ml/min; injection volume 3 ml; mobile phase A: 100% water, 0.1% TFA; mobile phase B: 100% ACN, 0.1% TFA; gradient elution from 0% B to 70% B in 70 min., detection 254 nm]. Fractions containing the desired compound were combined and concentrated in vacuum to provide the title compound as an off-white glass-like solid. Mass spec: Calculated 906.1. Observed 906.4.

### Example 4

### (R)—N-Methylnaltrexone 3-(N-methyl-N-(2-N-acetylarginylamino))ethylcarbamate

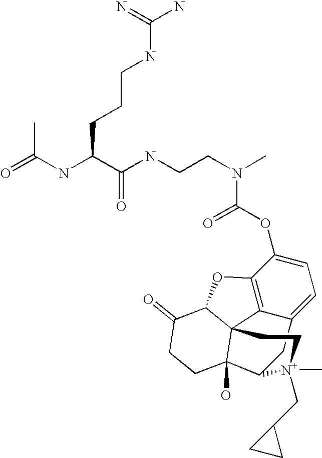

The product of Preparation 18 was dissolved in isopropanol (15 ml) and evaporated in vacuum. The residue was dissolved in a mixture of 5% m-cresol/TFA (10 ml). The reaction mixture was maintained at ambient temperature for 1 h followed by dilution with ethyl ester (100 ml). The formed precipitate was centrifuged, and the supernatant was discharged (procedure was repeated twice). The precipitate was dissolved in water (5 ml) and subjected to HPLC purification. [Nanosyn-Pack Microsorb (100-10) C-18 column (50×300 mm); flow rate=100 ml/min; injection volume 3 ml; mobile phase A: 100% water, 0.1% TFA; mobile phase B: 100% ACN, 0.1% TFA; gradient elution from 0% B to 70% B in 70 min., detection 254 nm]. Fractions containing the desired compound were combined and concentrated in vacuum. The residue was dissolved in isopropanol (15 ml) and evaporated in vacuum to provide a TFA salt of the title compound as colorless glass-like solid. The title compound was dissolved in dioxane (2 ml), and 4 M HCl/dioxane (10 ml) was added. The reaction mixture was maintained at ambient temperature for 30 min. Solvents were evaporated. The residue was dried in vacuum to provide a hydrochloric salt of the title compound (118 mg, 24%) as white solid. Yield: 118 mg (24%). Purity: 100%. Mass spec: Calculated 654.8. Observed 654.4.

### Preparation 19

### [2-(Boc-Asn)-aminoethyl]methylcarbamic acid benzyl ester

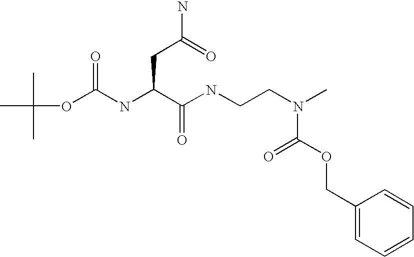

A solution of Boc-Asn-OH (464 mg, 2 mmol), (2-aminoethyl)methylcarbamic acid benzyl ester hydrochloride (537 mg, 2.2 mmol), BOP (1.06 g, 2.4 mmol), HOBt (329 mg, 2.4 mmol) and DIEA (1.11 ml, 6.4 mmol) in DMF (3 ml) was stirred at ambient temperature for 30 min followed by dilution with ethyl acetate (150 ml). The solution was washed with water (50 ml×3) and brine (50 ml). The organic layer was dried over MgSO₄ and evaporated to provide the title compound (820 mg, 95%) as yellowish oil. Mass spec: Calculated 423.5. Observed 423.4.

### Preparation 20

### N-(Boc-Asn)-N′-methyl-ethane-1,2-diamine

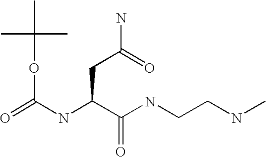

The product of Preparation 19 (820 mg, 1.94 mmol) was dissolved in methanol (50 ml) followed by the addition of Pd/C (5% wt, 500 mg) suspension in water (2 ml). The reaction mixture was subjected to hydrogenation (Parr apparatus, 60 psi) at ambient temperature for 30 min. The catalyst was filtered and washed with methanol. The filtrate was evaporated with isopropanol (2×20 ml) in vacuum to provide the title compound (540 mg, 97%) as colorless oil. Mass spec: Calculated 289.4. Observed 289.4.

### Preparation 21

### [2-(2-Amino-3-carbamoylpropionylamino)ethyl]methylcarbamic acid N-MTX ester

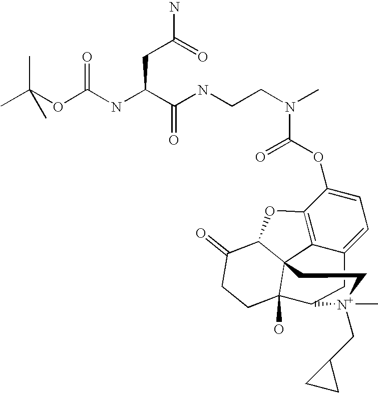

A suspension containing the TFA salt of N-MTX (470 mg, 1.0 mmol) and DIEA (174 μl, 1.0 mmol) in chloroform (4 ml) was sonicated in an ultrasonic bath at room temperature for 30 min followed by the addition of 4-nitrophenyl chloroformate (202 mg, 1.0 mmol). The reaction was sonicated in an ultrasonic bath at room temperature for 30 additional minutes followed by the addition of a solution of the product of Preparation 20 (461 mg, 1.6 mmol) and 1-hydroxybenzo-triazole (192 mg, 1.4 mmol) in DMF (3 ml). The reaction mixture was stirred overnight at ambient temperature. Volatile solvents were evaporated in vacuum. The residual solution was subjected to HPLC purification. [Nanosyn-Pack Microsorb (100-10) C-18 column (50×300 mm); flow rate=100 ml/min; injection volume 3 ml; mobile phase A: 100% water, 0.1% TFA; mobile phase B: 100% ACN, 0.1% TFA; gradient elution from 0% B to 70% B in 70 min., detection 254 nm]. Fractions containing the desired compound were combined and concentrated in vacuum to provide the title compound as an off-white glass-like solid. Mass spec: Calculated 670.8. Observed 670.6.

### Example 5

### (R)—N-Methylnaltrexone 3-(N-methyl-N-(2-N-asparaginylamino))ethylcarbamate

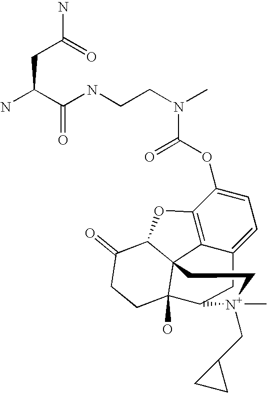

The product of Preparation 21 was dissolved in isopropanol (15 ml) and evaporated in vacuum. The residue was dissolved in dioxane (2 ml), and 4 M HCl/dioxane (10 ml) was added. The reaction mixture was maintained at ambient temperature for 30 min. Solvents were evaporated. The residue was dried in vacuum to provide a hydrochloric salt of the title compound as a white solid. Yield: 78 mg (13%). Purity: 95%. Mass spec: Calculated 570.7. Observed 570.4.

### Example 6

### (R)—N-Methylnaltrexone 3-(N-methyl-N-(2-N-aspartinylamino))ethylcarbamate

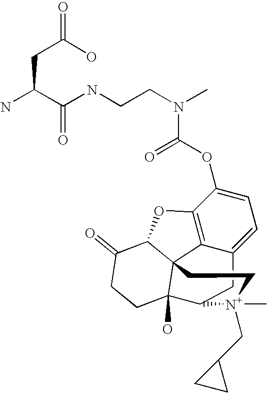

Prepared following the methods of Preparations 19-21 and Example 5. This method provided a hydrochloric salt of the title compound as a white solid. Yield: 52 mg (9%). Purity: 97%. Mass spec: Calculated 571.3. Observed 571.2.

### Example 7

### (R)—N-Methylnaltrexone 3-(N-methyl-N-(2-N-tyrosinylamino))ethylcarbamate

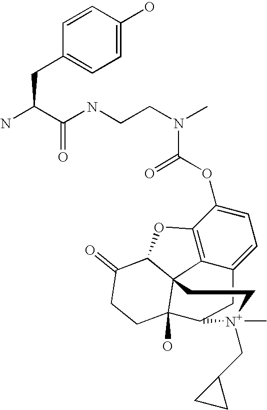

Prepared following the methods of Preparations 19-21 and Example 5. This method provided a hydrochloric salt of the title compound as a white solid. Yield: 68 mg (11%). Purity: 97%. Mass spec: Calculated 619.3. Observed 619.6.

### Example 8

### (R)—N-Methylnaltrexone 3-(N-methyl-N-(2-N-alaninylamino))ethylcarbamate

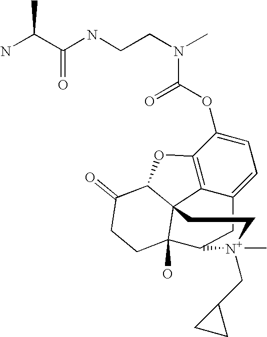

Prepared following the methods of Preparations 19-21 and Example 5. This method provided a hydrochloric salt of the title compound as a white solid. Yield: 105 mg (19%). Purity: 97%. Mass spec: Calculated 527.3. Observed 527.2.

### Example 9

### (R)—N-Methylnaltrexone 3-(N-methyl-N-(2-N-lysinylamino))ethylcarbamate

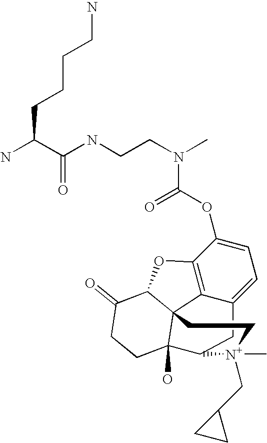

Prepared following the methods of Preparations 19-21 and Example 5. This method provided a hydrochloric salt of the title compound as a white solid. Yield: 104 mg (17%). Purity: 94%. Mass spec: Calculated 584.3. Observed 584.4.

### Preparation 22

### Synthesis of {2-[Boc-Gly-Arg(Pbf)]-aminoethyl}-methyl-carbamic acid benzyl ester

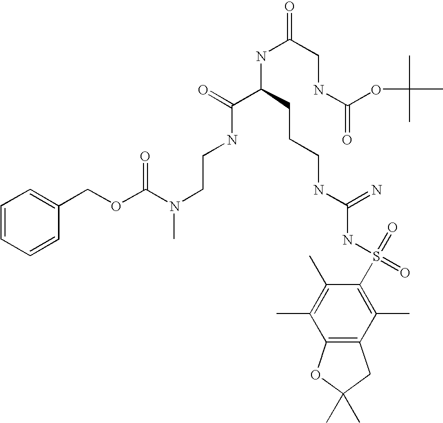

A solution of the product of Preparation 12 (1.19 g, 1.93 mmol), Boc-Gly-OH (406 mg, 2.32 mmol), BOP (1.44 g, 3.25 mmol) and DIEA (1.29 ml, 7.42 mmol) in DMF (3 ml) was maintained at ambient temperature for 30 min followed by dilution with ethyl acetate (100 ml). The solution was washed with water (30 ml×3) and brine (30 ml). The organic layer was dried over MgSO₄ and evaporated to provide the title compound (1.36 g, 91%) as yellowish oil. Mass spec: Calculated 775.0. Observed 774.9.

### Preparation 23

### Synthesis of N—[Boc-Gly-Arg(Pbf)]-N′-methyl-ethane-1,2-diamine

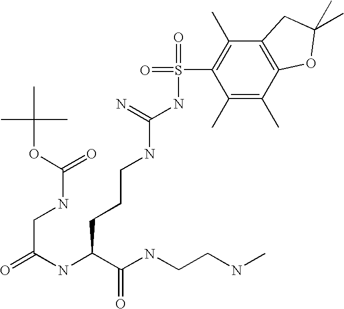

The product of Preparation 22 (1.36 g, 1.76 mmol) was dissolved in methanol (50 ml) followed by the addition of Pd/C (5% wt, 1 g) suspension in water (2 ml). The reaction mixture was subjected to hydrogenation (Parr apparatus, 60 psi) at ambient temperature for 30 min. The catalyst was filtered and washed with methanol. The filtrate was evaporated in vacuum to provide the title compound (1091 mg, 97%) as colorless oil. Mass spec: Calculated 640.8. Observed 640.3.

### Preparation 24

### Protected N-MTX Derivative

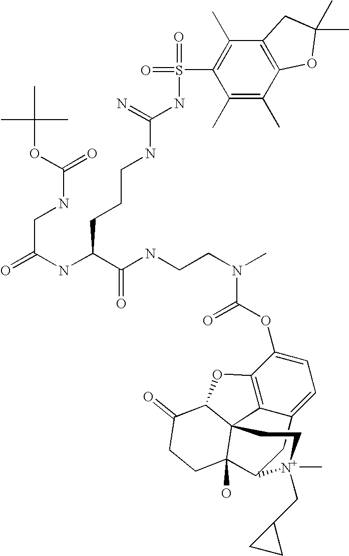

A suspension containing the TFA salt of N-MTX (329 mg, 0.7 mmol) and DIEA (122 μl, 0.7 mmol) in chloroform (4 ml) was sonicated in an ultrasonic bath at room temperature for 30 min followed by the addition of 4-nitrophenyl chloroformate (141 mg, 0.7 mmol). The reaction was sonicated in an ultrasonic bath at room temperature for 30 additional minutes followed by the addition of a solution of the product of Preparation 23 (511 mg, 0.8 mmol) and 1-hydroxybenzotriazole (164 mg, 1.2 mmol) in DMF (3 ml). The reaction mixture was stirred overnight at ambient temperature. Volatile solvents were evaporated in vacuum. The residual solution was subjected HPLC purification. [Nanosyn-Pack Microsorb (100-10) C-18 column (50×300 mm); flow rate=100 ml/min; injection volume 3 ml; mobile phase A: 100% water, 0.1% TFA; mobile phase B: 100% ACN, 0.1% TFA; gradient elution from 0% B to 70% B in 70 min., detection 254 nm]. Fractions containing the desired compound were combined and concentrated in vacuum to provide the title compound as off-white glass-like solid. Mass spec: Calculated 1021.3. Observed 1021.7.

### Example 10

### (R)—N-Methylnaltrexone 3-(N-methyl-N-(2-N-glycinylarginylamino))ethylcarbamate

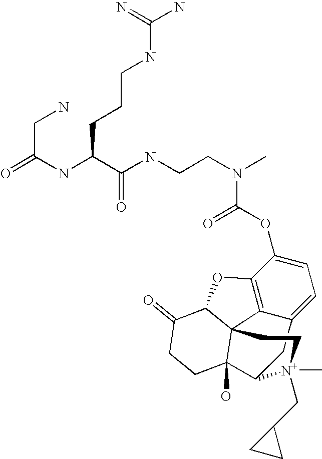

The product of Preparation 24 was dissolved in isopropanol (15 ml) and evaporated in vacuum. The residue was dissolved in a mixture of 5% m-cresol/TFA (10 ml). The reaction mixture was maintained at ambient temperature for 1 h followed by dilution with ethyl ester (100 ml). The formed precipitate was centrifuged, and the supernatant was discharged (procedure was repeated twice). The precipitate was dissolved in water (5 ml) and subjected to HPLC purification. [Nanosyn-Pack Microsorb (100-10) C-18 column (50×300 mm); flow rate=100 ml/min; injection volume 3 ml; mobile phase A: 100% water, 0.1% TFA; mobile phase B: 100% ACN, 0.1% TFA; gradient elution from 0% B to 70% B in 70 min., detection 254 nm]. Fractions containing desired compound were combined and concentrated in vacuum. The residue was dissolved in isopropanol (15 ml) and evaporated in vacuum to provide a TFA salt of the title compound as colorless glass-like solid. The solid was dissolved in dioxane (2 ml) and 4 M HCl/dioxane (10 ml) was added. The reaction mixture was maintained at ambient temperature for 30 min. Solvents were evaporated. The residue was dried in vacuum to provide a hydrochloric salt of the title compound as white solid. Yield: 85 mg (17%). Purity: 98%. Mass spec: Calculated 669.8. Observed 669.2.

### Example 11

### (R)—N-Methylnaltrexone 3-(N-methyl-N-(2-N—(N-acetyl)glycinylarginylamino))-ethylcarbamate

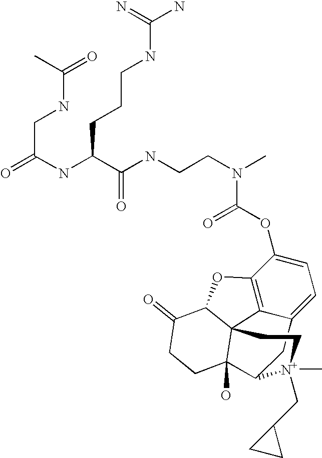

The title compound was prepared following the method described in Preparations 22 to 24 and Example 10, except that a Boc-protected acetylated glycine was used. The resulting residue was dried in vacuum to provide a hydrochloric salt of the title compound as white solid. Yield: 97 mg (17%). Purity: 97.3. Mass spec: Calculated 711.8. Observed 711.2.

### Preparation 25

### [2-(Boc-Ala)-aminoethyl]methylcarbamic acid benzyl ester

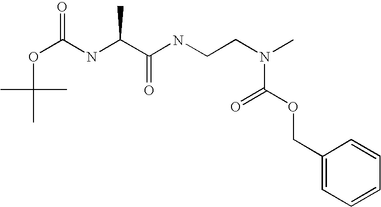

A solution of Boc-Ala-OH (380 mg, 2.0 mmol), (2-amino-ethyl)-methyl-carbamic acid benzyl ester hydrochloride (512 mg, 2.1 mmol), BOP (1.63 g, 2.4 mmol), and DIEA (1.11 ml, 6.4 mmol) in DMF (3 ml) was stirred at ambient temperature for 45 min followed by dilution with ethyl acetate (100 ml). The solution was extracted with water 3 times (30 ml each) and brine (30 ml). The organic layer was dried over MgSO₄ and evaporated to provide the title compound as a yellowish oil. Mass spec: Calculated 380.5. Observed 380.1.

### Preparation 26

### [2-(H-Ala)-aminoethyl]-methyl-carbamic acid benzyl ester

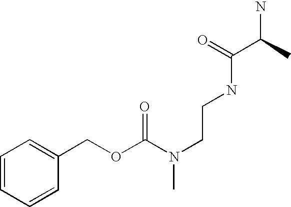

A solution of the product of Preparation 25 (2.0 mmol) dioxane (5 ml) was treated with 4 M HCl/dioxane (15 ml) at ambient temperature for 1 h. The solvent was evaporated. The residue was dried in vacuum overnight to provide hydrochloric salt of the title compound (599 mg, 95%) as off-white solid. Mass spec: Calculated 280.3. Observed 280.6.

### Preparation 27

### [2-(Boc-Gly-Ala)-aminoethyl}-methyl-carbamic acid benzyl ester

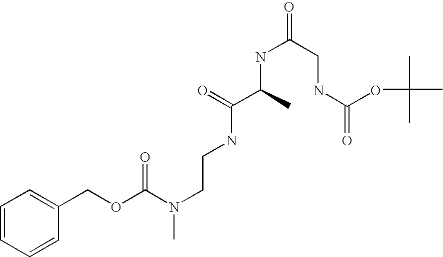

A solution of the product of Preparation 26 (599 mg, 1.78 mmol), Boc-Gly-OH (350 mg, 2.0 mmol), BOP (974 mg, 2.2 mmol) and DIEA (1.11 ml, 6.4 mmol) in DMF (3 ml) was maintained at ambient temperature for 30 min followed by the dilution with ethyl acetate (100 ml). The solution was extracted with water 3 times (30 ml each) and brine (30 ml). The organic layer was dried over MgSO₄ and evaporated to provide the title compound (759 mg, 98%) as yellowish oil. Mass spec: Calculated 437.5. Observed 437.2.

### Preparation 28

### Synthesis of N-(Boc-Gly-Ala)-N′-methyl-ethane-1,2-diamine

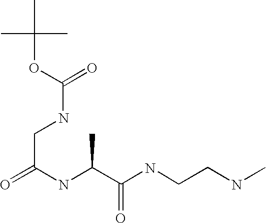

The product of Preparation 27 (759 mg, 1.74 mmol) was dissolved in methanol (50 ml) followed by the addition of Pd/C (5% wt, 1 g) suspension in water (2 ml). The reaction mixture was subjected to hydrogenation (Parr apparatus, 60 psi) at ambient temperature for 30 min. The catalyst was filtered and washed with methanol. The filtrate was evaporated in vacuum to provide the title compound (519 mg, 99%) as colorless oil. Mass spec: Calculated 303.4. Observed 303.7.

### Preparation 29

### Protected N-MTX Derivative

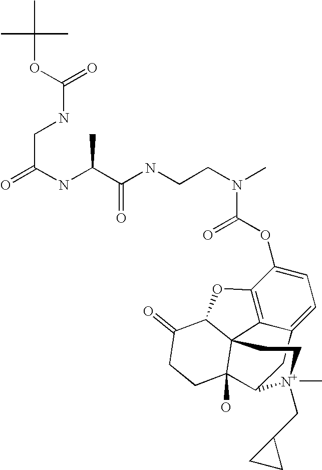

A suspension containing the TFA salt of N-MTX (376 mg, 0.8 mmol) and DIEA (140 μl, 0.8 mmol) in chloroform (4 ml) was sonicated on an ultrasonic bath at room temperature for 30 min followed by the addition of 4-nitrophenyl chloroformate (162 mg, 0.8 mmol). The reaction was sonicated on an ultrasonic bath at room temperature for an additional 30 min followed by the addition of a solution of the product of Preparation 28 (302 mg, 1.0 mmol) and HOBt (164 mg, 1.2 mmol) in DMF (3 ml). The reaction mixture was stirred overnight at ambient temperature. Volatile solvents were evaporated in vacuum. The residual solution was subjected HPLC purification. [Nanosyn-Pack Microsorb (100-10) C-18 column (50×300 mm); flow rate=100 ml/min; injection volume 3 ml; mobile phase A: 100% water, 0.1% TFA; mobile phase B: 100% ACN, 0.1% TFA; gradient elution from 0% B to 70% B in 70 min., detection 254 nm]. Fractions containing the desired compound were combined and concentrated in vacuum to provide the title compound as off-white glass-like solid. Mass spec: Calculated 684.8. Observed 684.6.

### Example 12

### (R)—N-Methylnaltrexone 3-(N-methyl-N-(2-N-glycinylalaninylamino))-ethylcarbamate

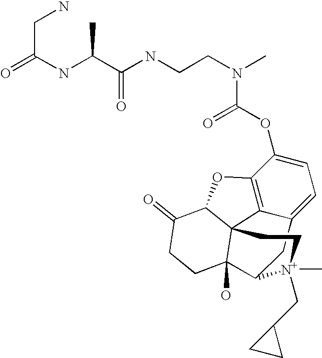

The product of Preparation 29 was dissolved in isopropanol (15 ml) and evaporated in vacuum. The residue was dissolved in dioxane (2 ml) and 4 M HCl/dioxane (10 ml) was added. The reaction mixture was maintained at ambient temperature for 30 min. Solvents were evaporated. The residue was dried in vacuum to provide a hydrochloric salt of the title compound as white solid. Yield: 58 mg (12%). Purity: 99.8%. Mass spec: Calculated 584.7. Observed 584.4.

### Example 13

### (R)—N-Methylnaltrexone 3-(N-methyl-N-(2-N-glycinylphenylalaninylamino))-ethylcarbamate

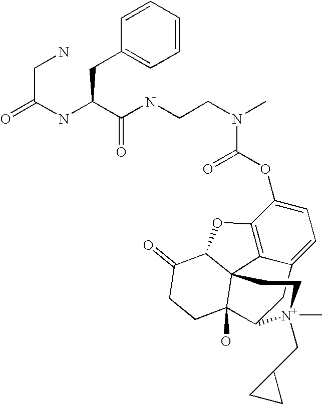

Prepared following the methods of Preparations 25-29 and Example 12. This method provided a hydrochloric salt of the title compound as white solid. Yield: 57 mg (12%). Purity: 100%. Mass spec: Calculated 660.8. Observed 660.4.

### Preparation 30

### 3-O-Isobutyryl-Naloxone

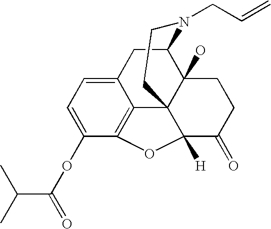

To a solution of naloxone hydrochloride dihydrate (1.75 g, 4.38 mmol) in anhydrous THF (120 ml) at 0° C. was added triethylamine (2.02 g, 20 mmol. After the reaction had been stirred for 15 min at 0° C., isobutyryl chloride (2.13 g, 20 mmol) was added dropwise. The reaction mixture was stirred at 0° C. for 30 min, then at ambient temperature for 2 h, before being quenched with a saturated solution of sodium bicarbonate (100 ml). The reaction mixture was extracted with DCM (2×100 ml), dried over MgSO₄, and evaporated to provide 1.58 g (91%) of crude title product. Mass spec: Calculated 397.5. Observed 398.2.

### Preparation 31

### 3-O-Isobutyryl-N-Methylnaloxone

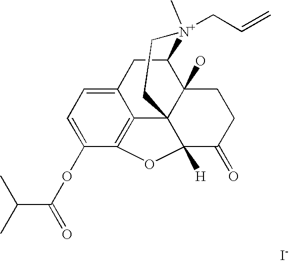

A suspension of 3-O-isobutyryl-naloxone (1.58 g, 3.97 mmol) in methyl iodide (MeI) (30 ml) in a 100 ml vial was frozen in liquid nitrogen until all of the methyl iodide froze. The vial was then vacuumed, filled with nitrogen gas, and then vacuumed. Methyl iodide was allowed to melt, and the procedure was repeated one more time. After the reaction mixture was heated for 18 h at a temperature of 90° C., the reaction mixture was checked by LCMS. Analysis indicated there was about 50% conversion of initial compound.

After evaporating the volatiles, the reaction mixture was suspended in water (300 ml) and heated at 90° C. for 30 min, filtered off, and water mother liquid extracted with DCM 3 times (30 ml each) to remove all of the starting material in the water solution. The precipitate and DCM-fractions were combined, and evaporated to give 0.57 g of starting material. The water solution was evaporated to give 1.21 g (88%) of crude quaternary iodide salt. Mass spec: Calculated 412.5. Observed 412.5.

### Preparation 32

### N-Methylnaloxone

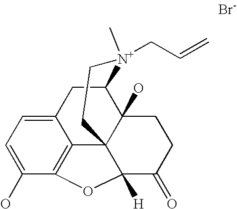

3-O-isobutyryl-N-methylnaloxone (1.21 g, 2.2 mmol) was dissolved in a mixture of 25 ml methanol and 25 ml water followed by the addition of 3 ml of 48% hydrogen bromide. The reaction mixture was heated with stirring at 50° C. overnight. Solvents were evaporated under reduced pressure. The residual oil was dissolved in a small amount of methanol. The formed precipitate was filtrated and dried in vacuum (0.69 g, 74%).

After HPLC purification, the resulting TFA salt of N-methylnaloxone was dissolved in DCM (10 ml), and 2 M HCl solution in diethyl ether (4 ml) was added. The mixture evaporated, the solid was dissolved in methanol (10 ml), and a 2 M HCl solution in diethyl ether (4 ml) was added. The mixture was evaporated and dried in high vacuum to yield a chloride salt of N-methylnaloxone. Yield: 0.60 g (72%). Mass spec: Calculated 342.4. Observed 342.5.

### Preparation 33

### 3-O-Acyl-Naltrexone

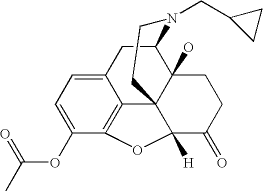

To a solution of naltrexone hydrochloride (1.0 g, 2.64 mmol) in anhydrous THF (200 ml) at 0° C. was added triethylamine (0.75 g, 5.8 mmol). After the reaction was stirred for 15 min at 0° C., acetyl chloride (0.42 g, 5.3 mmol) was added dropwise. The reaction mixture was stirred at 0° C. for 30 min, then at ambient temperature for 2 h, before being quenched with a saturated solution of sodium bicarbonate (150 ml). The reaction mixture was extracted with DCM 3 times (100 ml each), dried (MgSO₄), and evaporated to give 0.93 g (92%) of crude product. Mass spec: Calculated 383.4. Observed 384.1.

### Preparation 34

### 3-O-Acyl-Nalmefene

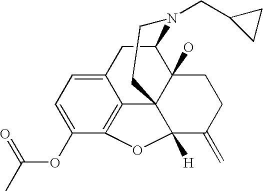

To a cooled (0° C.) solution of Preparation 33 (0.93 g, 2.43 mmol) in THF (20 ml) was added Tebbe's reagent dropwise (9.8 ml, 4.9 mmol). The reaction mixture was allowed to warm to room temperature for 10 min, diluted with methanol and with water. The reaction mixture was used in the next step without work-up.

### Preparation 35

### Nalmefene

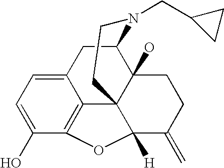

The reaction mixture from Preparation 34 was acidified with 1 N HCl aq. solution and heated for 2 h at 50° C. The reaction mixture was basified with ammonia solution, with further extraction with DCM 3 times (30 ml each). DCM fractions were dried (MgSO₄), evaporated and purified by HPLC. The TFA salt of nalmefene was converted into free amine by means of sodium bicarbonate, extracted with DCM 3 times (30 ml each), dried (MgSO₄), and evaporated. Yield 0.32 g (39%). Mass spec: Calculated 339.4. Observed 340.4.

### Preparation 36

### 3-O-Isobutyryl-Nalmefene

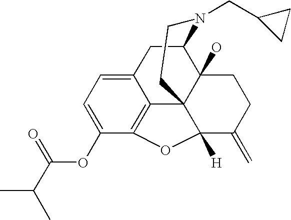

To a solution of Preparation 35 (0.32 g, 0.94 mmol) in anhydrous THF (100 ml) at 0° C. was added triethylamine (0.2 g, 2.0 mmol). After the reaction was stirred for 15 min at 0° C., isobutyryl chloride (0.213 g, 2.0 mmol) was added dropwise. The reaction mixture was stirred at 0° C. for 30 min, then at ambient temperature for 2 h, before being quenched with a saturated solution of sodium bicarbonate (100 ml). The reaction mixture was extracted with DCM 3 times (30 ml each), dried (MgSO₄), and evaporated to give 0.34 g (89%) of crude product. Mass spec: Calculated: 409.5. Observed 411.

### Preparation 37

### 3-O-Isobutyril-N-Methylnalmefene Iodide

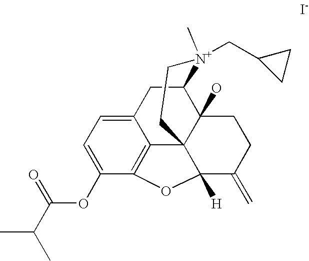

A solution of the product of Preparation 36 (0.34 g, 0.83 mmol) in methyl iodide (20 ml) was stirred at 85° C. for 100 h. After evaporating volatiles, the reaction mixture was suspended in water (200 ml) and extracted with benzene (3×50 ml) to remove all starting material in the water solution. The water solution was evaporated to give 0.41 g (89%) of crude quaternary iodine salt. Mass spec: Calculated 424. Observed 424.8.

### Preparation 38

### N-Methylnalmefene

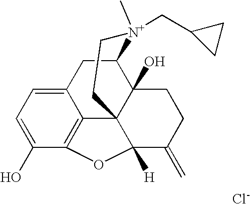

A solution of the product of Preparation 37 (0.41 g, 0.74 mmol) in water (100 ml) with 1 N HCl aq. solution (20 ml) was stirred at 40° C. for 120 h. The reaction mixture was lyophilized to remove water and yielded a chloride salt of N-methylnalmefene. Yield 0.27 g (92%). Mass spec: Calculated 354.5. Observed 354.8.

### Preparation 39

### 3-(4-Nitrophenyl)-N-Methylnaloxone Carbonate

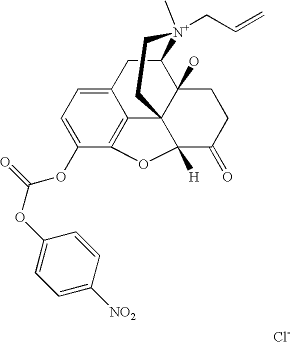

An HCl salt of N-methylnaloxone (0.174 g, 0.46 mmol) (Preparation 32) and DIEA (0.065 g, 0.51 mmol) were suspended in a mixture of 0.5 ml DMF and 10 ml MeCN using an ultrasound bath. Nitrophenyl chloroformate (0.103 g, 0.51 mmol) was added to the reaction mixture which was then sonicated for 2 h at room temperature. The resulting solution of 3-(4-Nitrophenyl)-N-methylnaloxone carbonate was used in next step without work-up.

### Preparation 40

### Protected Derivative of N-Methylnaloxone

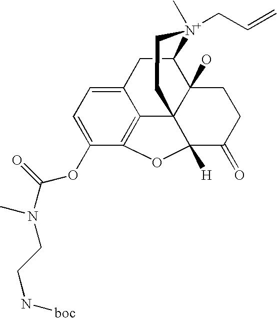

To the suspension of the product of Preparation 39 (0.23 g, 0.46 mmol) were added boc-N-methylethyldiamine (0.088 g, 0.5 mmol) and HOBT (0.067 g, 0.5 mmol). The reaction mixture was stirred overnight. Volatiles were evaporated under vacuum and the resulting oil was purified by HPLC to give 0.19 g (78%) of the title compound.

### Example 14

### N-Methylnaloxone 3-(N-methyl-N-(2-aminoethyl))carbamate

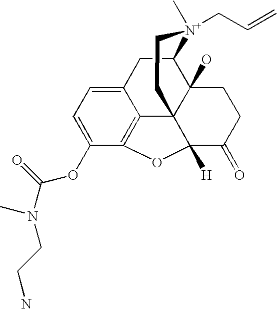

A solution of the product of Preparation 40 (0.19 g, 0.35 mmol) in 10 ml dioxane and 4 M solution of HCl in dioxane (0.8 ml, 3.2 mmol) was stirred at ambient temperature for 8 h and further stirred at 27° C. overnight to produce the crude title compound, which was purified by HPLC. To a solution of the TFA salt of the title compound (about 0.35 mmol) in DCM was added a 2 M HCl solution in ether (0.5 ml). After 10 min of stirring at room temperature, the reaction mixture was evaporated at reduced pressure. The procedure was repeated one more time. Drying under high vacuum yielded a chloride salt of the title compound. Yield: 0.064 g (38%). Purity: 99.6%. Mass spec: Calculated 442.5. Observed 442.9.

### Preparation 41

### 3-(4-Nitrophenyl)-N-methylnalmefene Carbonate

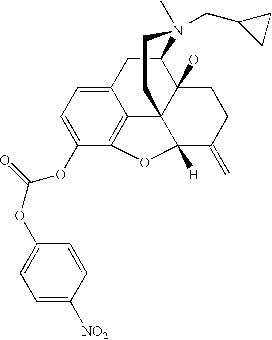

A TFA salt of N-methylnalmefene (0.068 g, 0.14 mmol) (Preparation 38) and DIEA (0.02 g, 0.16 mmol) were suspended in a mixture of 0.5 ml DMF and 10 ml MeCN using an ultrasound bath. Nitrophenylchloroformate (0.032 g, 0.16 mmol) was added to reaction mixture. The reaction mixture was sonicated at room temperature for 1 h. The resulting solution of 3-(4-nitrophenyl)methylnalmefene carbonate was used in next step with out work-up.

### Preparation 42

### Protected Derivative of N-Methylnalmefene

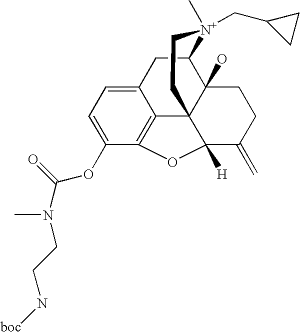

To the suspension of the product of Preparation 41 (0.072 g, 0.14 mmol) were added boc-N-methylethyldiamine (0.027 g, 0.154 mmol) and HOBT (0.021 g, 0.154 mmol). The reaction mixture was stirred overnight. Volatiles were evaporated under vacuum and the resulting oil was purified by HPLC to give 0.06 g (78%) compound 3.

### Example 15

### N-Methylnalmefene 3-(N-methyl-N-(2-aminoethyl))carbamate

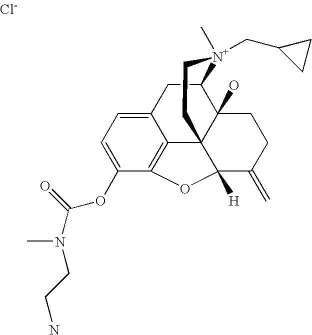

A solution of the product of Preparation 42 (0.19 g, 0.28 mmol) and TFA (0.32 g, 2.8 mmol) in 10 ml DCM was stirred at 28° C. overnight to produce the crude title compound. The reaction mixture was evaporated under high vacuum, and the crude title compound was purified by HPLC. To a solution of the TFA salt of the title compound (about 0.28 mmol) in DCM was added 2 M HCl solution in ether (0.28 ml). After 10 min of stirring at room temperature, the reaction mixture was evaporated at reduced pressure at ambient temperature. The procedure was repeated one more time. Drying under high vacuum yielded a chloride salt of the title compound. Yield: 0.104 g (75%). Purity: 98.3%. Mass spec: Calculated 454.6. Observed 454.9.

Following the methods of the Preparations and Examples described hereinabove, the following compounds may also be prepared, and are provided as further embodiments of the invention:—

### N-Methylnaltrexone (N-MTX) Structures

### NAc-Ala-N-MTX

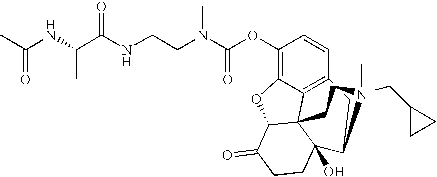

### NAc-Asn-N-MTX

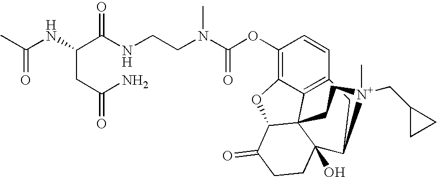

### NAc-Leu-N-MTX

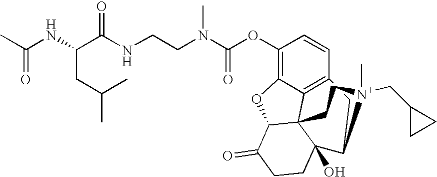

### NAc-Lys-N-MTX

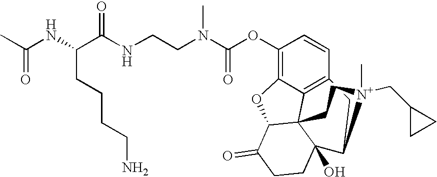

### NAc-Tyr-N-MTX

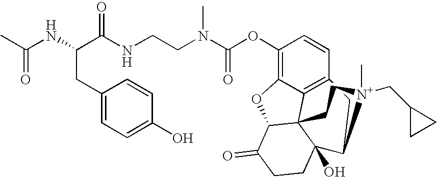

### N-Methylnaloxone Structures

### Ala-N-Methylnaloxone

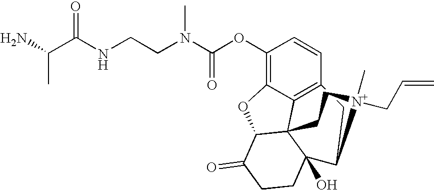

### NAc-Ala-N-Methylnaloxone

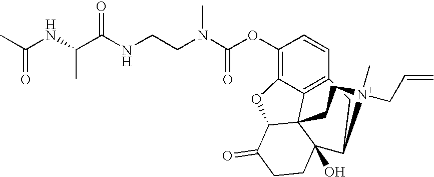

### Arg-N-Methylnaloxone

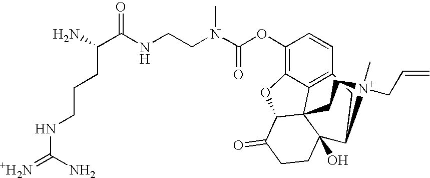

### NAc-Arg-N-Methylnaloxone

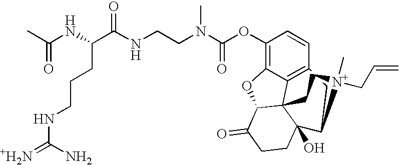

### Arg-Gly-N-Methylnaloxone

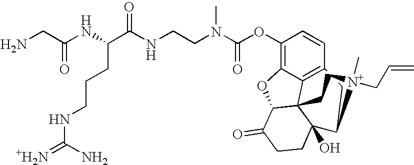

### NAc-Arg-Gly-N-Methylnaloxone

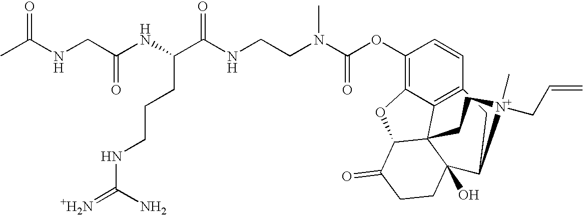

### Asn-N-Methylnaloxone

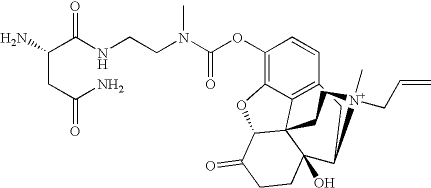

### NAc-Asn-N-Methylnaloxone

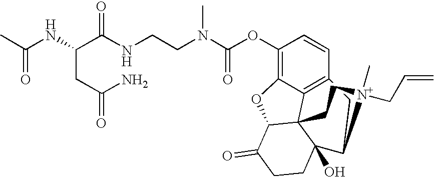

### Asp-N-Methylnaloxone

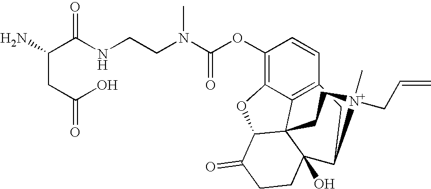

### NAc-Asp-N-Methylnaloxone

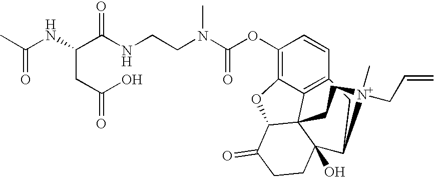

### Leu-N-Methylnaloxone

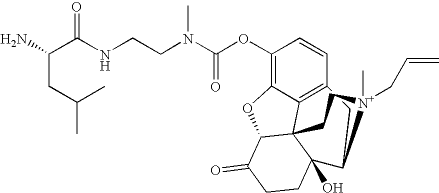

### NAc-Leu-N-Methylnaloxone

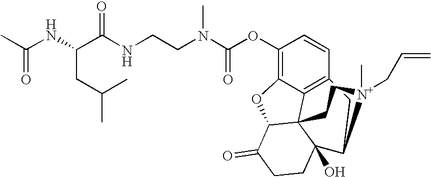

### Lys-N-Methylnaloxone

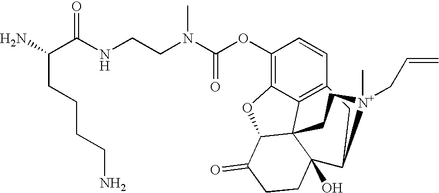

### NAc-Lys-N-Methylnaloxone

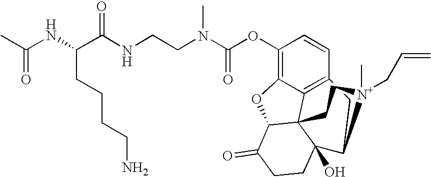

### Tyr-N-Methylnaloxone

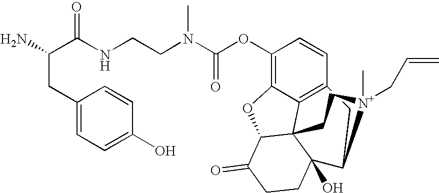

### NAc-Tyr-N-Methylnaloxone

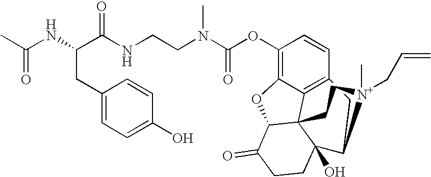

### N-Methyldiprenorphine Structures

### Ala-N-Methyldiprenorphine

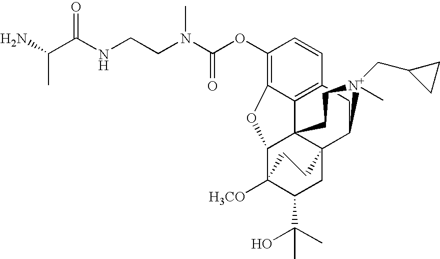

### NAc-Ala-N-Methyldiprenorphine

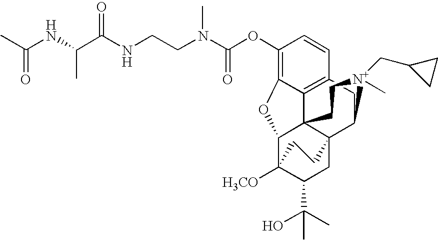

### Arg-N-Methyldiprenorphine

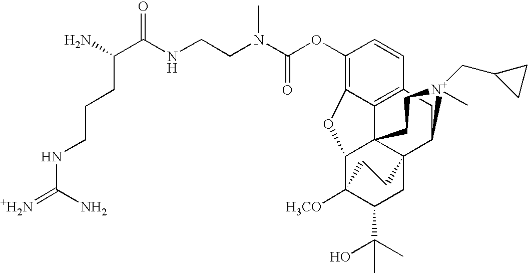

### NAc-Arg-N-Methyldiprenorphine

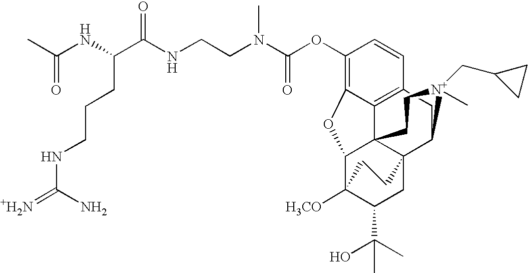

### Arg-Gly-N-Methyldiprenorphine

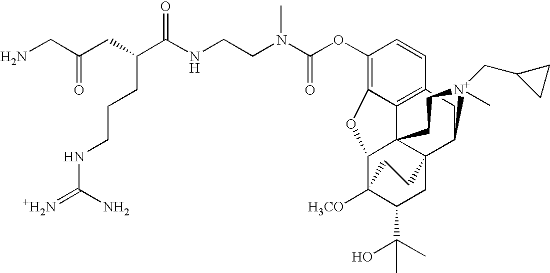

### NAc-Arg-Gly-N-Methyldiprenorphine

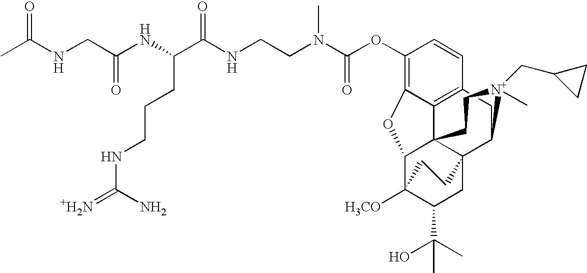

### Asn-N-Methyldiprenorphine

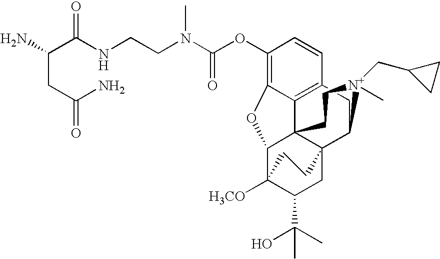

### NAc-Asn-N-Methyldiprenorphine

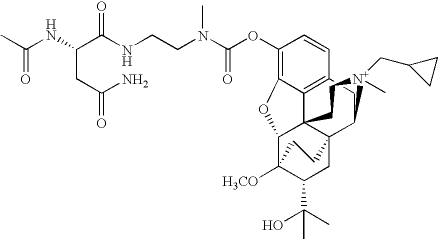

### Asp-N-Methyldiprenorphine

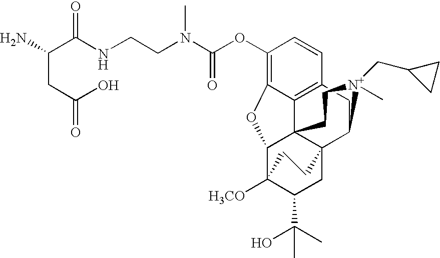

### NAc-Asp-N-Methyldiprenorphine

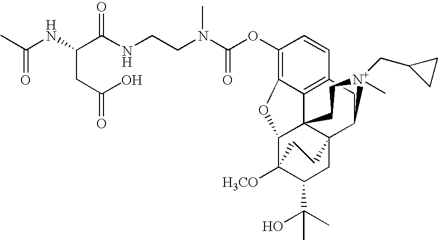

### Leu-N-Methyldiprenorphine

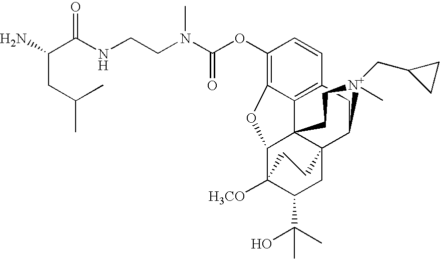

### NAc-Leu-N-Methyldiprenorphine

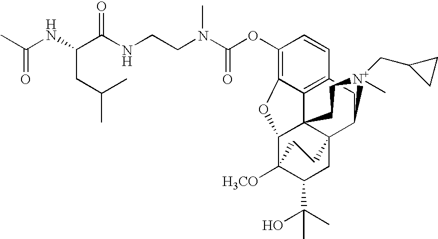

### Lys-N-Methyldiprenorphine

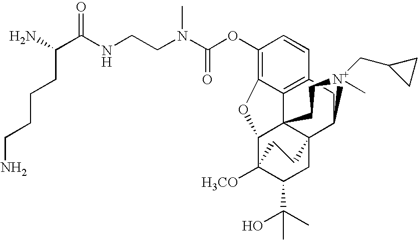

### NAc-Lys-N-Methyldiprenorphine

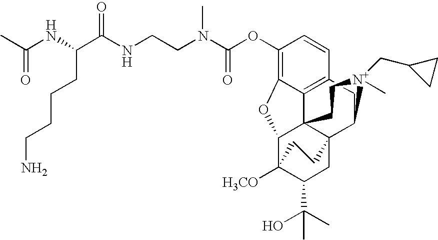

### Tyr-N-Methyldiprenorphine

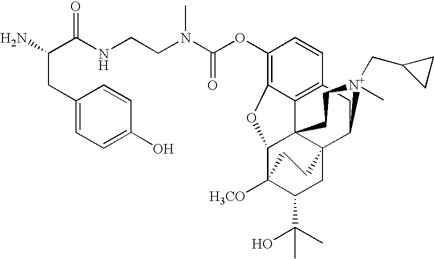

### NAc-Tyr-N-Methyldiprenorphine

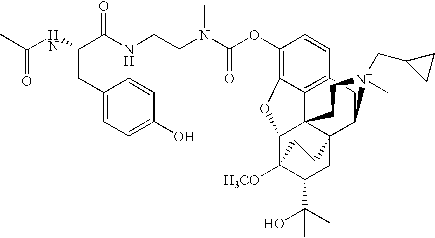

### N-Methylnalmefene Structures

### Ala-N-Methylnalmefene

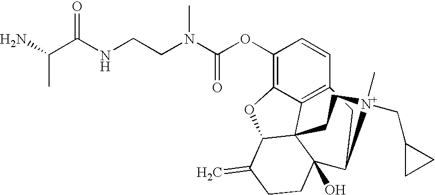

### NAc-Ala-N-Methylnalmefene

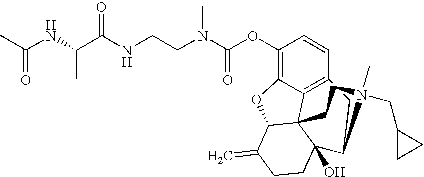

### Arg-N-Methylnalmefene

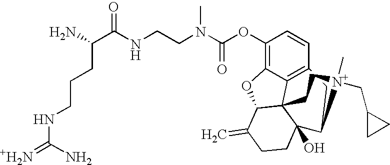

### NAc-Arg-N-Methylnalmefene

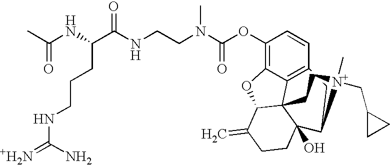

### Arg-Gly-N-Methylnalmefene

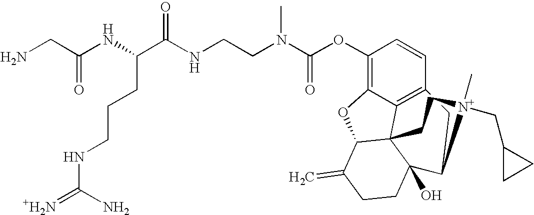

### NAc-Arg-Gly-N-Methylnalmefene

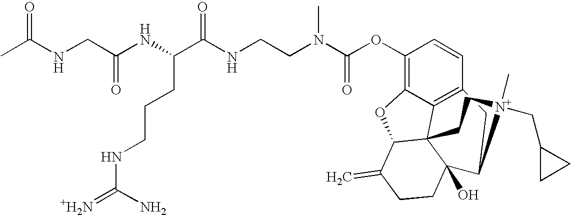

### Asn-N-Methylnalmefene

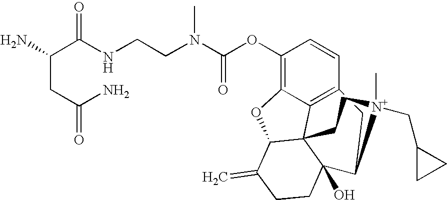

### NAc-Asn-N-Methylnalmefene

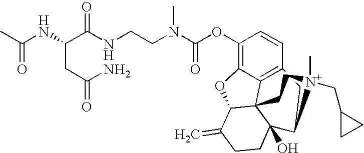

### Asp-N-Methylnalmefene

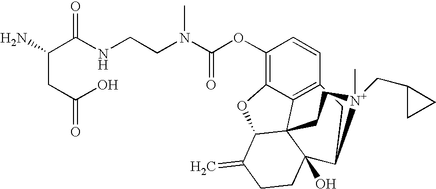

### NAc-Asp-N-Methylnalmefene

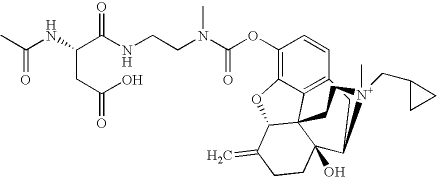

### Leu-N-Methylnalmefene

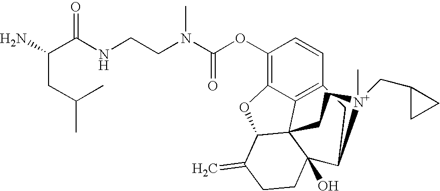

### NAc Leu-N-Methylnalmefene

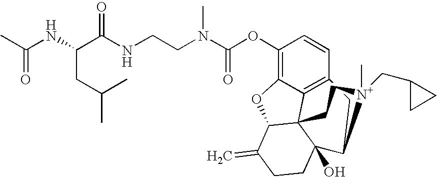

### Lys-N-Methylnalmefene

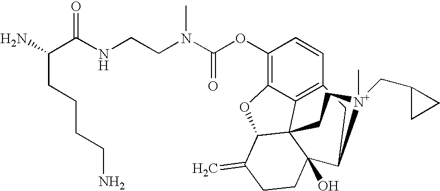

### NAc-Lys-N-Methylnalmefene

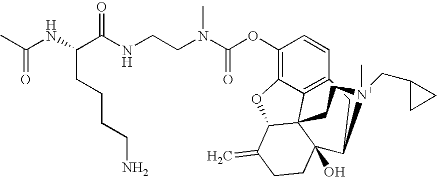

### Tyr-N-Methylnalmefene

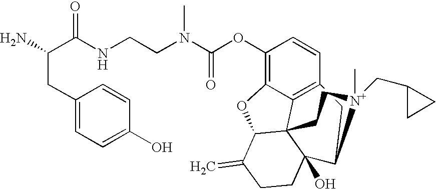

### NAc-Tyr-N-Methylnalmefene

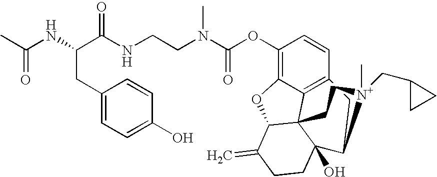

### N-Methyldiprenorphine 3-(N-methyl-N-(2-aminoethyl))carbamate

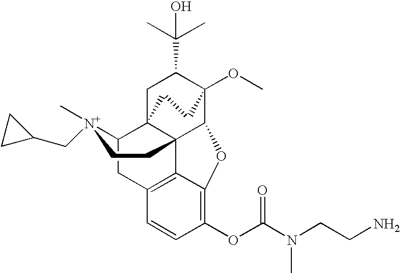

The counter-ion is conveniently a chloride ion.

### Protocols for Evaluating Test Compounds

1. Pharmacokinetic Data

Plasma Timecourse of Peripheral Phenolic Opioid Antagonists Following PO Administration to Rat

Oral dosing: The test compounds were dissolved in saline and dosed via oral gavage into jugular vein cannulated male Sprague-Dawley rats. N-MTX and N-MNLX, each at 20 mg/kg, were used as positive controls and the test compounds were dosed at the doses indicated in Tables 1 and 2. At specified time points, blood samples were withdrawn, quenched into methanol, centrifuged at 14000 rpm @ 4° C., and stored at −80° C. until analysis. Samples were quantified via LC/MS/MS using an ABI 3000 triple-quad mass spectrometer.

Results:

TABLE 1
Maximum concentration of (R)-N-methylnaltrexone (N-MTX)
found in blood after oral (P0) dosing in rats.

| Example | Dose (mg/kg) | Cmax N-MTX (ng/ml) |
|---|---|---|
| N-MTX | 20 | 8.8 |
| 1 | 20 | 72.5 |
| 2 | 20 | 31.5 |
| | 40 | 57.8 |
| 3 | 20 | 15.1 |
| 4 | 20 | ND |
| 5 | 20 | ND |
| 6 | 20 | 14.8 |
| 7 | 20 | ND |
| 8 | 12.68 | ND |
| 9 | 20 | ND |
| 10 | 20 | 11.3 |
| 11 | 20 | 5.5 |
| 12 | 20 | ND |
| 13 | 20 | 3.0 |

ND = not detected

TABLE 2
Maximum concentration of N-methylnaloxone (N-MNLX) found
in blood after oral (P0) dosing in rats.

| Example | Dose (mg/kg) | Cmax N-MNLX (ng/ml) |
|---|---|---|
| N-MNLX | 20 | 17.3 |
| 14 | 20 | 40.1 |

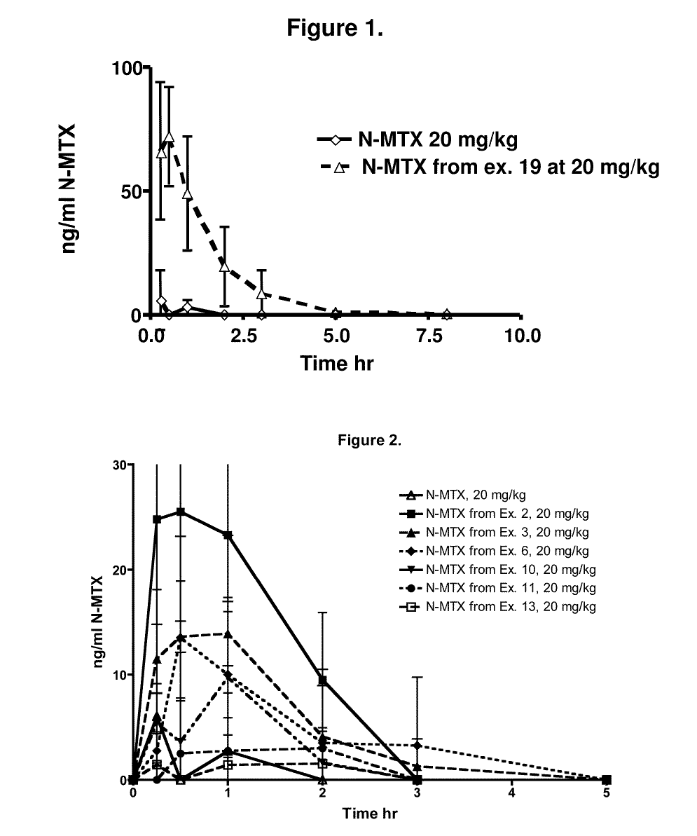

FIG. 1. Plasma concentration time course of the production of N-MTX following oral (PO) dosing in rats. The solid line represents the plasma concentration of N-MTX following PO dosing of N-MTX at 20 mg/kg. The dashed line represents the plasma concentration of N-MTX produced following oral dosing of Example 1 at 20 mg/kg.

FIG. 2. Plasma concentration time courses of the production of N-MTX following oral (PO) dosing in rats. The lines, as labelled, represent the plasma concentrations of N-MTX following PO dosing of Examples 2, 3, 6, 10, 11 and 13 respectively, each at 20 mg/kg

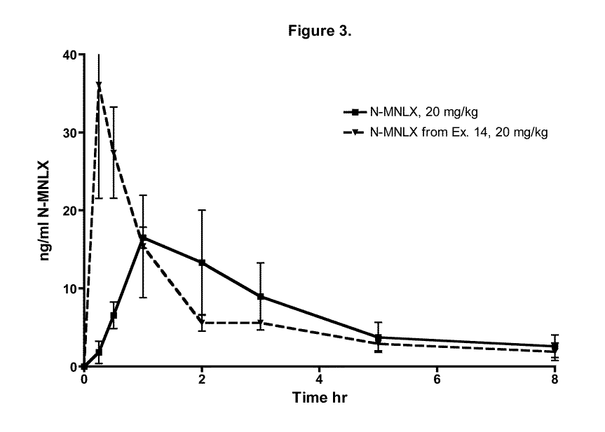

FIG. 3. Plasma concentration time course of the production of N-MNLX following oral (PO) dosing in rats. The solid line represents the plasma concentration of N-MNLX following PO dosing of N-MNLX at 20 mg/kg. The dashed line represents the plasma concentration of N-MNLX produced following oral dosing of Example 14 at 20 mg/kg.

By examining the Cmax values in Tables 1 and 2 and the plasma time courses represented by FIGS. 1, 2 and 3, it is clear that the utility of (R)—N-methylnaltrexone and N-methylnaloxone may be limited by their poor pharmacokinetic profiles (e.g. oral bioavailabilities). This limitation can be overcome by pro-drugs represented by the test compounds of Examples 1, 2, 3, 6, 10, 11, 13 and 14, each of which provides an improved pharmacokinetic profile (e.g. increased oral bioavailability). Specifically, oral administration of these test compounds leads to enhanced Cmax values and/or enhanced persistence of exposure as compared to the respective peripheral phenolic opioid antagonists from which they were derived. The doses used for the test compounds of Examples 4, 5, 7, 8, 9, and 12 did not afford detectable levels of N-MTX, but it is not believed that this result indicates that these test compounds are incapable of functioning as pro-drugs for N-MTX. These doses may have been too low for the specific model and/or analytical methods employed. It should be noted that the test compounds were dosed as mg/kg body weight, not mg equivalents of N-MTX or N-MNLX, respectively.

1. A method of antagonising peripheral action of an opioid in a patient undergoing opioid treatment, which comprises orally administering to said patient an effective amount of a compound of formula (I) 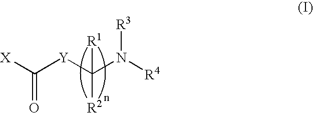
or a salt, hydrate or solvate thereof wherein:
X is a residue of a peripheral phenolic opioid antagonist, wherein the hydrogen atom of the phenolic hydroxyl group is replaced by a covalent bond to —C(O)—Y—(C(R¹)(R²))ₙ—N—(R³)(R⁴);
Y is —NR⁵— and R⁵ is (1-4C)alkyl;
n is an integer from 1 to 10;
each R¹, R², and R³ is independently hydrogen, alkyl, substituted alkyl, aryl or substituted aryl, or R¹ and R² together with the carbon to which they are attached form a cycloalkyl or substituted cycloalkyl group, or two R¹ or R² groups on adjacent carbon atoms, together with the carbon atoms to which they are attached, form a cycloalkyl or substituted cycloalkyl group;
R⁴ is hydrogen
or a derivative thereof capable of delivering the compound of formula (I) into the gut.

2. A method as claimed in claim 1, in which the peripheral phenolic opioid antagonist is (R)—N-methylnaltrexone, N-methylnaloxone, N-methyldiprenorphine or N-methylnalmefene.

3. A method as claimed in claim 1, in which the peripheral phenolic opioid antagonist is (R)—N-methylnaltrexone or N-methylnaloxone.

4. (canceled)

5. A method as claimed in claim 1, in which R⁵ is methyl.

6. A method as claimed in claim 1, in which n is 2 or 3.

7. A method as claimed in claim 1, in which R¹ and R² are each hydrogen.

8. A method as claimed in claim 1, in which R³ is hydrogen or (1-4C)alkyl.

9. A compound of structural Formula (I): 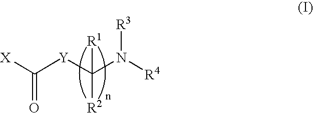
or a salt, hydrate or solvate thereof wherein:
X is (R)—N-methylnaltrexone, N-methylnaloxone, N-methyldiprenorphine or N-methylnalmefene wherein the hydrogen atom of the phenolic hydroxyl group is replaced by a covalent bond to —C(O)—Y—(C(R¹)(R²))ₙ—N—(R³)(R⁴);
Y is —NR⁵—, —O— or —S—;
n is an integer from 1 to 10;
each R¹, R², R³ and R⁵ is independently hydrogen, alkyl, substituted alkyl, aryl or substituted aryl, or R¹ and R² together with the carbon to which they are attached form a cycloalkyl or substituted cycloalkyl group, or two R¹ or R² groups on adjacent carbon atoms, together with the carbon atoms to which they are attached, form a cycloalkyl or substituted cycloalkyl group; and
R⁴ is hydrogen.

10. A compound as claimed in claim 9, wherein X is (R)—N-methylnaltrexone or N-methylnaloxone.

11. A compound as claimed in claim 9 or claim 10, in which Y is NR⁵ and R⁵ is hydrogen or (1-4C)alkyl.

12. A compound as claimed in claim 11, in which R⁵ is methyl.

13. A compound as claimed in claim 9, in which n is 2 or 3.

14. A compound as claimed in claim 9, in which R¹ and R² are each hydrogen.

15. A compound as claimed in claim 9, in which R³ is hydrogen or (1-4C)alkyl.

16. A pharmaceutical composition, which comprises a compound as claimed in claim 9 and a pharmaceutically acceptable carrier.

17. (canceled)

18. A compound as claimed in claim 9, wherein X is (R)—N-methylnaltrexone.

19. A compound as claimed in claim 9, wherein X is N-methylnaloxone.

20. A compound as claimed in claim 9, wherein
X is (R)—N-methylnaltrexone, N-methylnaloxone, N-methyldiprenorphine or N-methylnalmefene wherein the hydrogen atom of the phenolic hydroxyl group is replaced by a covalent bond to —C(O)—Y—(C(R¹)(R²))ₙ—N—(R³)(R⁴);
Y is —NR⁵—;
R⁵ is (1-4C)alkyl;
R¹ and R² are independently selected from hydrogen and (1-4C)alkyl;
n is 2 or 3;
R³ is hydrogen or (1-4C)alkyl;
R⁴ is hydrogen.

21. A compound as claimed in claim 20, wherein X is (R)—N-methylnaltrexone.

22. A compound as claimed in claim 20, wherein X is N-methylnaloxone.
